(19) United States
(12) Patent Application Publication
(10) Pub. No.: US 2022/0218667 A1
(43) Pub. Date: Jul. 14, 2022

(54) DUAL INHIBITORS OF SOLUBLE EPOXIDE HYDROLASE AND METHODS OF USE THEREOF

(71) Applicant: OROX Biosciences, Inc., San Diego, CA (US)

(72) Inventor: Mehran F. MOGHADDAM, San Diego, CA (US)

(21) Appl. No.: 17/605,525

(22) PCT Filed: Apr. 24, 2020
(86) PCT No.: PCT/US2020/029899

§ 371 Date: Oct. 21, 2021

Related U.S. Application Data
(60) Provisional application No. 62/838,756, filed on Apr. 25, 2019.

(51) Int. Cl. A61K 31/415; A61P 11/00; A61K 45/06

(52) U.S. Cl. CPC A61K 31/415; A61K 45/06; A61P 11/00

(57) ABSTRACT

Disclosed herein are methods to treatment of inflammatory and fibrotic progression, for example, idiopathic pulmonary fibrosis (IPF) with a dual inhibitor of soluble epoxide hydrolase inhibitors (sEH) and a secondary target. In particular, the invention relates to treatment of fibrotic progression with a dual inhibitor of sEH and COX-2. In one embodiment, IPF is treated with 4-(5-phenyl-3-{3-[3-(4-trifluoromethyl-phenyl)-ureido]-propyl}-pyrazol-1-yl)-benzenesulfonamide (PTUPB) or a derivative thereof. Also disclosed herein are compositions useful in treatment of fibrotic progression.

### CROSS-REFERENCE

This application claims benefit of U.S. Provisional Application No. 62/838,756, filed on Apr. 25, 2019, which is herein incorporated by reference in its entirety.

### FIELD

The present disclosure relates generally to treatment of fibrotic progression with a dual inhibitor of soluble epoxide hydrolase (sEH) and a secondary target. In some embodiments, the disclosure relates generally to treatment of fibrotic progression via inhibition of inflammation with a dual inhibitor of soluble epoxide hydrolase (sEH) and a secondary target.

### BACKGROUND

Fibrotic progression occurs in various disease including fibrosis of the liver, heart, kidney, skin, intestine, and lung. Fibrosis of the lung is as known as pulmonary fibrosis. Pulmonary fibrosis can be caused by a number of different conditions, including sarcoidosis, hypersensitivity pneumonitis, collagen vascular disease, infections such as COVID-19, and inhalant exposure. In some cases, pulmonary fibrosis can be caused by inflammation. The diagnosis of these conditions can usually be made by careful history, physical examination, chest radiography, including a high-resolution computer tomographic scan (HRCT), and open lung or transbronchial biopsies. However, in a significant number of patients, no underlying cause for the pulmonary fibrosis can be found. These conditions of unknown etiology have been termed idiopathic interstitial pneumonias (IIP).

Idiopathic pulmonary fibrosis (IPF) is the most common form of IIP. IPF is a chronic and ultimately fatal disease characterized by a progressive decline in lung function. IPF has a low median survival of only 3 years from diagnosis. Depending on the particular clinical code, the prevalence of IPF in the US ranges from 14 to as high as 156 cases per 100,000 persons. (Raghu, G. et al. Am. J. Respir. Crit. Car. Med., 2006, 174:810-6.) As the pathogenesis of IPF is a function of several factors (inflammatory infiltrate, increased cytokines, cellular differentiation, etc.), a plethora of therapeutic approaches have been examined. Trials focused on modulating the underlying inflammation have either provided no significant benefit (e.g. N-Acetylcysteine) or have led to an increased number of adverse events (e.g., combination of Prednisone, Azathioprine, and N-Acetylcysteine). (Martinez, F. J. et al. NEJM 2014, 370:2093-101; Raghu, G. et al. NEJM, 2012, 366(21):1968-77.) Clinical approaches focused on additional aspects of IPF, such as, epithelial cell death, aberrant wound healing, fibroblast and myofibroblast differentiation, and extracellular matrix (ECM) remodeling have met with limited success. (Ahluwalia, N. et al. Am. J. Respir. Crit. Care Med. 2014, 190(8):867-78.)

The recent clinical approval of two small molecule therapeutics, Pirfenidone and Nintedanib, are a significant achievement for treating and/or preventing IPF, but neither Pirfenidone nor Nintedanib is curative for IPF. Thus, there is a long-felt yet unmet medical need for alternative therapies for fibrosis, fibrotic progression, IIP, and/or IPF. The disclosure provides such a novel therapy, and compositions and methods to address and solve this need.

### SUMMARY

Successful treatment or prevention of fibrotic progression and the underlying inflammatory process, including idiopathic interstitial pneumonias (IIP) and in particular idiopathic pulmonary fibrosis (IPF), can be challenging not only because the etiology of these disorders is often heterogeneous and poorly understood, but also because many treatment modalities have been tried and shown to be ineffective.

In one aspect, the present disclosure provides a method for treating or preventing a disease associated with fibrotic progression in a subject in need thereof, comprising administering a dual inhibitor of sEH and a secondary target. In one embodiment of the first aspect, the dual inhibitor is PTUPB (OX1) and the disease associated with fibrotic progression is idiopathic pulmonary fibrosis.

In a second aspect, the present disclosure provides PTUPB for use in the treatment of idiopathic pulmonary fibrosis. In a third aspect, the present disclosure provides a dual inhibitor capable of inhibiting sEH and a secondary target.

In some embodiments is a method for treating or preventing a disease associated with fibrotic progression in a subject in need thereof, comprising administering a dual inhibitor of sEH and a secondary target, wherein the secondary target is selected from the group consisting of FLT3, PDGFR-α, PDGFR-β, VEGFR-1, VEGFR-2, COX-2, 5-LOX, FGFR, TGF-β, and CCR1.

In some embodiments is a method for treating or preventing a disease associated with fibrotic progression in a subject in need thereof, comprising administering a dual inhibitor of sEH and a secondary target, wherein the dual inhibitor comprises a sEH pharmacophore selected from the group consisting of Ureas, Carbamates, Amides, and Pyrazoles. In some embodiments the sEH pharmacophore comprises the structure:

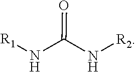

In some embodiments is a method for treating or preventing a disease associated with fibrotic progression in a subject in need thereof, comprising administering a dual inhibitor of sEH and a secondary target, wherein the dual inhibitor comprises any of the compounds disclosed in U.S. Pat. No. 9,096,532.

In some embodiments is a method for treating or preventing a disease associated with fibrotic progression in a subject in need thereof, comprising administering a dual inhibitor of sEH and a secondary target, wherein the secondary target is COX-2.

In some embodiments is a method for treating or preventing a disease associated with fibrotic progression in a subject in need thereof, comprising administering a dual inhibitor of sEH and a secondary target, wherein the dual inhibitor is a compound of Formula I:

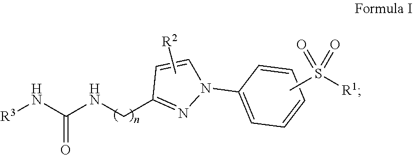

wherein:
R¹ is selected from the group consisting of C₁₋₆ alkyl, —NR^{1a}R^{1b} and cycloalkyl;
R^{1a} and R^{1b} are each independently selected from the group consisting of H and C₁₋₆ alkyl;
R² is selected from the group consisting of C₁₋₆ alkyl, cycloalkyl and aryl, wherein the cycloalkyl and aryl are each optionally substituted with C₁₋₆ alkyl;
R³ is selected from the group consisting of cycloalkyl and aryl, each optionally substituted with from 1 to 3 R^{3a} groups wherein each R^{3a} is independently selected from the group consisting of C₁₋₆ alkyl, C₁₋₆ alkoxy, halogen, C₁₋₆ haloalkyl, and C₁₋₆ haloalkoxy; subscript n is an integer from 0 to 6;
and salts and optical isomers thereof.

In some embodiments is a method for treating or preventing a disease associated with fibrotic progression in a subject in need thereof, comprising administering a dual inhibitor of sEH and a secondary target, wherein the dual inhibitor is a compound of Formula Ia:

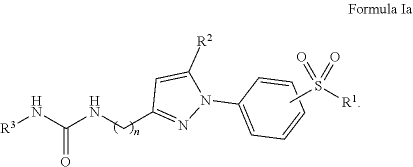

In some embodiments of the compounds of Formula I or Ia, R³ is phenyl optionally substituted with 1 to 3 R^{3a} groups wherein each R^{3a} is independently selected from the group consisting of C₁₋₆ alkyl, C₁₋₆ alkoxy, halogen, C₁₋₆ haloalkyl, and C₁₋₆ haloalkoxy. In some embodiments of the compounds of Formula I or Ia, R³ is phenyl optionally substituted with 1 R^{3a} group wherein R^{3a} is selected from the group consisting of C₁₋₆ alkyl, C₁₋₆ alkoxy, halogen, C₁₋₆ haloalkyl, and C₁₋₆ haloalkoxy. In some embodiments of the compounds of Formula I or Ia, R³ is

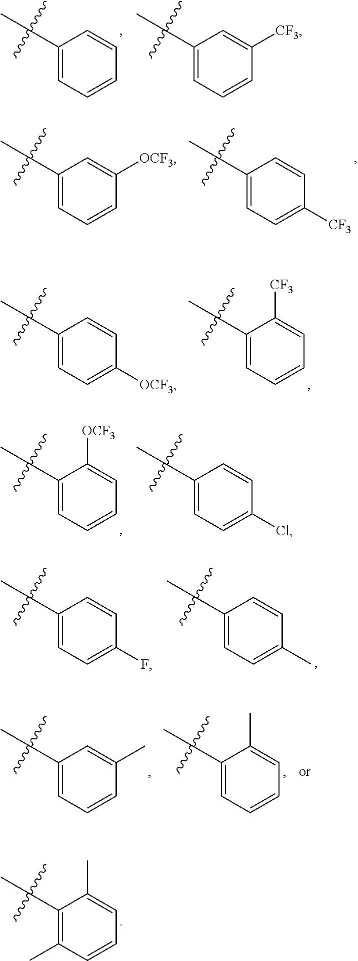

In some embodiments of the compounds of Formula I or Ia. In some embodiments of the compounds of Formula I or Ia, R³ is

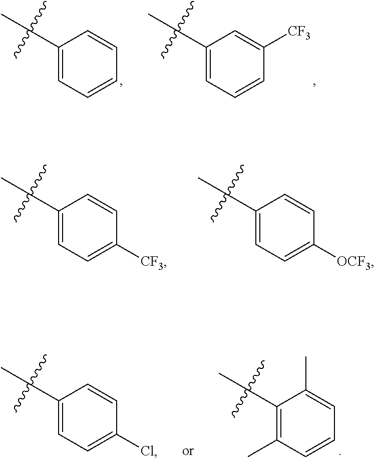

In some embodiments of the compounds of Formula I or Ia, R³ is cycloalkyl optionally substituted with 1 to 3 R^{3a} groups wherein each R^{3a} is independently selected from the group consisting of C₁₋₆ alkyl, C₁₋₆ alkoxy, halogen, C₁₋₆ haloalkyl and C₁₋₆ haloalkoxy. In some embodiments of the compounds of Formula I or Ia, R³ is unsubstituted cycloalkyl. In some embodiments of the compounds of Formula I or Ia, R³ is

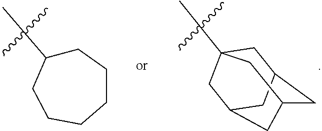 [SMILES: *C1CCCCCC1 ; *C12CC3CC(CC(C3)C1)C2]

In some embodiments of the compounds of Formula I or Ia, R² is phenyl optionally substituted with C₁₋₆ alkyl. In some embodiments of the compounds of Formula I or Ia, R² is unsubstituted phenyl. In some embodiments of the compounds of Formula I or Ia, R¹ is —NR^{1a}R^{1b}. In some embodiments of the compounds of Formula I or Ia, R¹ is —NH₂. In some embodiments of the compounds of Formula I or Ia, R¹ is C₁₋₆ alkyl. In some embodiments of the compounds of Formula I or Ia, R¹ is —CH₃. In some embodiments of the compounds of Formula I or Ia, n is an integer from 1 to 3. In some embodiments of the compounds of Formula I or Ia, n is 1. In some embodiments of the compounds of Formula I or Ia, n is 2. In some embodiments of the compounds of Formula I or Ia, n is 3.

In some embodiments is a method for treating or preventing a disease associated with fibrotic progression in a subject in need thereof, comprising administering a dual inhibitor of sEH and a secondary target, wherein the dual inhibitor is PTUPB (OX1).

In some embodiments is a method for treating or preventing a disease associated with fibrotic progression in a subject in need thereof, comprising administering a dual inhibitor of sEH and a secondary target, wherein the disease associated with fibrotic progression is idiopathic pulmonary fibrosis. In some embodiments is a method for treating or preventing an inflammatory disease associated with fibrotic progression in a subject in need thereof, comprising administering a dual inhibitor of sEH and a secondary target, wherein the disease associated with fibrotic progression is idiopathic pulmonary fibrosis.

In some embodiments is PTUPB for use in the treatment of idiopathic pulmonary fibrosis.

In some embodiments is a dual inhibitor capable of inhibiting sEH and a secondary target, wherein the dual inhibitor inhibits sEH with IC₅₀ of 1 micromolar or less, 100 nanomolar or less, 50 nanomolar or less, 10 nanomolar or less, 1 nanomolar or less, 50 picomolar or less, 10 picomolar or less, or 1 nanomolar or less. In some embodiments is a dual inhibitor capable of inhibiting sEH and a secondary target, wherein the dual inhibitor inhibits the secondary target with IC₅₀ of 1 micromolar or less, 100 nanomolar or less, 50 nanomolar or less, 10 nanomolar or less, 1 nanomolar or less, 50 picomolar or less, 10 picomolar or less, or 1 nanomolar or less. In some embodiments is a dual inhibitor capable of inhibiting sEH and a secondary target, wherein the dual inhibitor has a half-life in liver microsomes of at least five minutes, at least one hour, at least two hours, at least twelve hours, at least one day, at least two days, or at least one week.

In some embodiments is a method for treating or preventing a disease associated with fibrotic progression in a subject in need thereof, comprising administering a compound whose structure comprises an inhibitor of sEH. In some embodiments is a method for treating or preventing a disease associated with fibrotic progression in a subject in need thereof, comprising administering a compound whose structure comprises an inhibitor of sEH, wherein the inhibitor of sEH is selected from the group consisting of PTUPB (OX1), Pirfenidone, Nintedanib, and GSK225629415 (OX3). In some embodiments is a method for treating or preventing a disease associated with fibrotic progression in a subject in need thereof, comprising administering a compound whose structure comprises an inhibitor of sEH, wherein the inhibitor of sEH is selected from the small molecules disclosed in US 2009/0023731. In some embodiments is a method for treating or preventing a disease associated with fibrotic progression in a subject in need thereof, comprising administering a compound whose structure comprises an inhibitor of sEH, wherein the inhibitor of sEH is selected from the small molecules disclosed in U.S. Pat. No. 8,815,951.

The methods and compositions of the present disclosure address the need for treatment or prevention of fibrotic progression by providing alternative methods of treating fibrotic progression, IIP, and IPF. In particular, dual inhibitors of sEH and secondary targets reflect a treatment modality technically distinguishable from Pirfenidone and Nintedanib.

### DESCRIPTION OF DRAWINGS

FIG. 1 depicts body weights measured over 21 days (Mean±SEM). The mean weight of negative control animals (n=5) increased by ˜6% compared to their average starting weight, while that of the bleomycin-treated mice (n=10) decreased by ˜10% in the course of this experiment. Nintedanib, Pirfenidone, and OX3-treated mice (n=10) were similar to the bleomycin-treated control mice with regards to body weight loss. However, OX1-treated mice (n=10) started to recover after week 1 and exhibited an average net gain of ˜1% compared to the starting point at the end of the experiment.

FIG. 2 depicts lung weight at termination (Mean±SEM). A. Actual lung weights. Lungs were harvested from each animal after sacrifice on day 21 and weighed before removal of BALF. B. Lung weight index. Lung weights for each animal was corrected for the corresponding body weight to yield “Lung Weight Index.”

FIG. 3A-3D depict cell counts in mouse BALF (Mean±SEM). No Bleomycin and Bleomycin treated groups behaved as expected with respect to this parameter. FIG. 3A shows total Leukocyte Count in BALF. FIG. 3B shows absolute number of macrophages in BALF. FIG. 3C shows absolute number of neutrophils in BALF. FIG. 3D shows absolute number of lymphocytes in BALF.

FIG. 4 depicts soluble collagen concentrations in BALF (Mean±SEM). OX1 was the most effective test article in lowering production of soluble collagen in mouse lung BALF.

FIG. 5A-5C depict lung histology scores (Mean±SEM). FIG. 5A depicts mouse lung, average Ashcroft scores. FIG. 5B depicts mean increased collagen (Fibrosis) scores. FIG. 5C depicts mean inflammation scores.

### DETAILED DESCRIPTION

The present disclosure relates to treating fibrotic progression, such as fibrotic progression due to pulmonary fibrosis, or in particular, idiopathic interstitial pneumonias (IIP), or more particularly, idiopathic pulmonary fibrosis (IPF) with inhibitors of soluble epoxide hydrolase (sEH). In some embodiments, the present disclosure relates to treating fibrotic progression due to pulmonary inflammation and fibrosis.

Epoxide hydrolases are a family of proteins that mediate the addition of water to both exogenous and endogenous epoxides, resulting in epoxide ring opening to diols. This enzyme family consists of soluble epoxide hydrolase (sEH), microsomal epoxide hydrolase (mEH), cholesterol epoxide hydrolase and leukotriene A4 (LTA4) epoxide hydrolase. Mono-oxidation of arachidonic acid, physiologically catalyzed by cytochrome P450s (CYPs), leads to a class of epoxides referred to as epoxyeicosatrienoic acids (EETs). Soluble epoxide hydrolase functions as a metabolizing enzyme for EETs leading to hydration of the epoxide and generation of the corresponding dihydroxyeicosatrienoic acids (DHETs). Inhibition of sEH has led to increased concentrations of EETs in several animal models resulting in beneficial effects. Several studies in sEH-knockout mice have demonstrated that lack of sEH led to protection against both experimental cerebral ischemia, and cardiac reperfusion injury, and diminished inflammatory activities such as inflammatory bowel disease.

One inhibitor of soluble epoxide hydrolase (sEH), TPPU, has been shown to provide protective effects against inflammatory fibrosis, although no clinical data has yet been disclosed on TPPU. (Zhou et al. Cell Tissue Res. 2016 February; 363(2): 399-409) The structure of TPPU is:

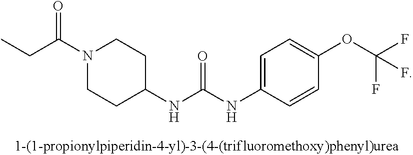 [SMILES: CCC(=O)N1CCC(NC(=O)Nc2ccc(OC(F)(F)F)cc2)CC1]

In some embodiments, the inhibitor of soluble epoxide hydrolase (sEH) has the structure:

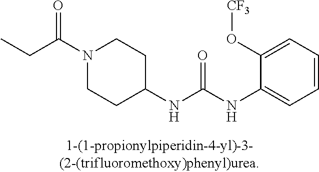 [SMILES: CCC(=O)N1CCC(NC(=O)Nc2ccccc2OC(F)(F)F)CC1]

Another sEH inhibitor, GSK2256294 (OX3) is in clinical trials for a pulmonary disorder that is etiologically distinct from fibrotic progression or IPF, namely chronic obstructive pulmonary disease (COPD). (Lazaar et al. Br J Clin Pharmacol. 2016 May; 81(5):971-9.) But GSK2256294 has never been reported to be useful for IPF. The structure of GSK2256294 is:

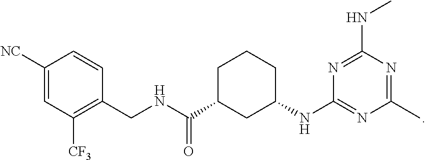 [SMILES: CNc1nc(C)nc(N[C@H]2CCC[C@@H](C(=O)NCc3ccc(C#N)cc3C(F)(F)F)C2)n1]

4-(5-phenyl-3-{3-[3-(4-trifluoromethyl-phenyl)-ureido]-propyl}-pyrazol-1-yl)-benzenesulfonamide (PTUPB) is dual inhibitor of sEH and COX-2 (Hwang et al. Synthesis and Structure-Activity Relationship Studies of Urea-Containing Pyrazoles as Dual Inhibitors of Cyclooxygenase-2 and Soluble Epoxide Hydrolase. J Med Chem. 2011 Apr. 28; 54(8): 3037-3050). It is a derivative of the COX-2 inhibitor celecoxib and of the sEH inhibitor trans-AUCB. Celecoxib, sold under the brand-name CELEBREX™, has the structure:

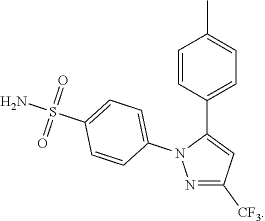 [SMILES: Cc1ccc(-c2cc(C(F)(F)F)nn2-c2ccc(S(N)(=O)=O)cc2)cc1]

Celecoxib has been shown to induce exacerbation of asthma in some patients. (See Subramanian et al. Celecoxib Induced Asthma Exacerbation. Chest. October 2015 Volume 148, Issue 4, Supplement, Page 403A.

Trans-AUCB is a very potent inhibitor of sEH (IC50=0.5 nM). It displays high oral bioavailability for doses ranging from 0.01 to 1 mg/kg. It has the structure:

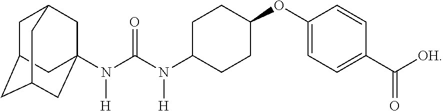

PTUPB has the structure:

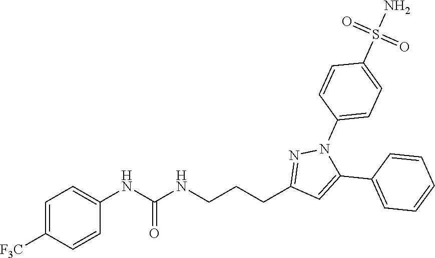 [SMILES: NS(=O)(=O)c1ccc(-n2nc(CCCNC(=O)Nc3ccc(C(F)(F)F)cc3)cc2-c2ccccc2)cc1]

### 4-(5-phenyl-3-(3-(3-(4-(trifluoromethyl)phenyl)ureido)propyl)-1H-pyrazol-1-yl)benzenesulfonamide

PTUPB is representative of a class of compounds sharing the sEH pharmacophore:

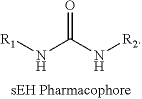

Other sEH pharmacophores include amide, urea, carbamate, and pyrazole moieties. Examples of compounds useful in the present methods are provided by US 2009/0023731, U.S. Pat. Nos. 8,815,951, and 7,951,831, which are each incorporated by reference herein in their entirety.

More specifically, PTUPB is a member of a genus of compounds having the structure of Formula I.

Various analogs of PTUPB, which act as dual inhibitors of sEH and COX-2 are described in U.S. Pat. No. 9,096,532, which provides compounds and compositions, e.g., a series of compounds wherein a 1,5-biarylpyrazole group is conjugated to a urea group by a non-cleavable covalent chain, that are useful as dual COX-2/sEH inhibitors. U.S. Pat. No. 9,096,532 is incorporated by reference herein in its entirety.

### Definitions

The abbreviations used herein have their conventional meaning within the chemical and biological arts.

The abbreviations used herein have their conventional meaning within the chemical and biological arts.

Where substituent groups are specified by their conventional chemical formulae, written from left to right, they equally encompass the chemically identical substituents that would result from writing the structure from right to left, e.g., —CH₂O— is equivalent to —OCH₂—.

As used herein, the term “alkyl” refers to a straight or branched, saturated, aliphatic radical having the number of carbon atoms indicated. For example, C₁-C₆ alkyl includes, but is not limited to, methyl, ethyl, propyl, butyl, pentyl, hexyl, iso-propyl, iso-butyl, sec-butyl, tert-butyl, etc.

As used herein, the term “alkoxy” refers to an alkyl radical as described above which also bears an oxygen substituent capable of covalent attachment to another hydrocarbon for example, methoxy, ethoxy or t-butoxy group.

As used herein, the term “halogen,” by itself or as part of another substituent, means, unless otherwise stated, a fluorine, chlorine, bromine, or iodine atom.

As used herein, the term “haloalkyl” refers to alkyl as defined above where some or all of the hydrogen atoms are substituted with halogen atoms. Halogen (halo) preferably represents chloro or fluoro, but may also be bromo or iodo. For example, haloalkyl includes trifluoromethyl, fluoromethyl, 1,2,3,4,5-pentafluoro-phenyl, etc. The term “perfluoro” defines a compound or radical which has at least two available hydrogens substituted with fluorine. For example, perfluorophenyl refers to 1,2,3,4,5-pentafluorophenyl, perfluoromethane refers to 1,1,1-trifluoromethyl, and perfluoromethoxy refers to 1,1,1-trifluoromethoxy.

As used herein, the term “haloalkoxy” refers to alkoxy as defined above where some or all of the hydrogen atoms are substituted with halogen atoms. “Haloalkoxy” is meant to include monohaloalkyl(oxy) and polyhaloalkyl(oxy).

As used herein, the term “cycloalkyl” refers to a saturated or partially unsaturated, monocyclic, fused bicyclic or bridged polycyclic ring assembly containing from 3 to 12 ring atoms, or the number of atoms indicated. For example, C₃-C₈ cycloalkyl includes cyclopropyl, cyclobutyl, cyclopentyl, cyclohexyl, cycloheptyl, and cyclooctyl. Cycloalkyl also includes norbornyl and adamantyl.

As used herein, the term “aryl” means, unless otherwise stated, a polyunsaturated, aromatic, hydrocarbon substituent which can be a single ring or multiple rings (preferably from 1 to 3 rings) which are fused together or linked covalently. Examples include, but are not limited to, phenyl, biphenyl, naphthyl, and benzyl.

Substituents for the aryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R″′ are independently selected from hydrogen, C₁-C₈ alkyl and heteroalkyl, unsubstituted aryl and heteroaryl, (unsubstituted aryl)-(C₁-C₄)alkyl, and (unsubstituted aryl)oxy-(C₁-C₄)alkyl.

As used herein, the phrases “pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors and colors, and the like. One of skill in the art will recognize that other pharmaceutical excipients are useful in the present invention.

As used herein, the phrase “soluble epoxide hydrolase” (sEH) refers to an enzyme which in endothelial, smooth muscle and other cell types converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The amino acid sequence of human sEH is also set forth as SEQ ID NO:2 of U.S. Pat. No. 5,445,956; the nucleic acid sequence encoding the human sEH is set forth as nucleotides 42-1703 of SEQ ID NO:1 of that patent. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)).

As used herein, the terms “cyclooxygenase” and “COX” refers to an enzyme that is associated with the formation of biological mediators called prostanoids, i.e., prostaglandin biosynthesis. Inhibition of COX is associated with relief from the symptoms of inflammation and pain. The terms “COX-1” and “COX-2” refer to two exemplary cyclooxygenase enzymes, i.e., isozymes of COX. COX-1 and COX-2 differ from each other in their regulation of expression and tissue distribution.

As used herein, the term “contacting” refers to the process of bringing into contact at least two distinct species such that they can react. It should be appreciated, however, the resulting reaction product can be produced directly from a reaction between the added reagents or from an intermediate from one or more of the added reagents which can be produced in the reaction mixture.

As used herein, the phrase “therapeutically effective amount” refers to an amount of a conjugated functional agent or of a pharmaceutical composition useful for treating or ameliorating an identified disease or condition, or for exhibiting a detectable therapeutic or inhibitory effect. The effect can be detected by any assay method known in the art.

As used herein, the terms “treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

As used herein, the term “modulate” refers to the ability of a compound to increase or decrease the function, or activity, of the associated activity (e.g., soluble epoxide hydrolase, i.e. sEH. “Modulation”, as used herein in its various forms, is meant to include antagonism and partial antagonism of the activity associated with sEH.

As used herein, the terms “patient” or “subject” refers to a living organism suffering from or prone to a condition that can be treated by administration of a pharmaceutical composition as provided herein. Non-limiting examples include humans, other mammals and other non-mammalian animals.

As used herein the term “inhibiting,” refers to the partial or total blocking of the function or associated activity. Inhibitors of sEH are compounds that bind to, partially or totally block the sEH enzyme's activity.

As used herein, the term “reduce or inhibit,” when used in reference to angiogenesis, means that the amount of new blood vessel formation that occurs in the presence of an antagonist is decreased below the amount of blood vessel formation that occurs in the absence of an exogenously added antagonist. The terms “reduce” and “inhibit” are used together because it is recognized that the amount of angiogenesis can be decreased below a level detectable by a particular assay method and, therefore, it may not be possible to determine whether angiogenesis is reduced to a very low level or completely inhibited.

The terms “a,” “an,” or “a(n)”, when used in reference to a group of substituents or “substituent group” herein, mean at least one. For example, where a compound is substituted with “an” alkyl or aryl, the compound is optionally substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, or physiological conditions.

### Compounds

In some embodiments provided herein a compound of Formula I:

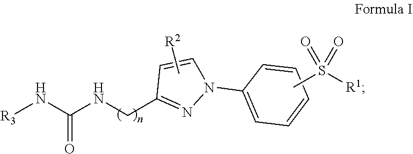

wherein R¹ is C₁₋₆ alkyl, —NR^{1a}R^{1b} or cycloalkyl; R^{1a} and R^{1b} are each independently selected from the group consisting of H and C₁₋₆ alkyl; R² is C₁₋₆ alkyl, cycloalkyl or aryl, wherein the cycloalkyl and aryl are each optionally substituted with C₁₋₆ alkyl; R³ is cycloalkyl or aryl, each optionally substituted with from 1 to 3 R^{3a} groups wherein each R^{3a} is independently selected from C₁₋₆ alkyl, C₁₋₆ alkoxy, halogen, C₁₋₆ haloalkyl or C₁₋₆ haloalkoxy; and subscript n is an integer from 0 to 6. Also included are the salts and isomers of a compound of Formula I.

In some embodiments provided herein is a compound of Formula Ia:

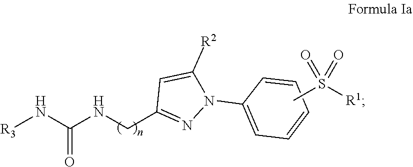

In some embodiments provided herein is a compound of Formula Ib:

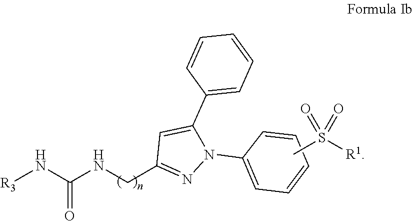

In some embodiments provided herein is a compound of Formula Ic:

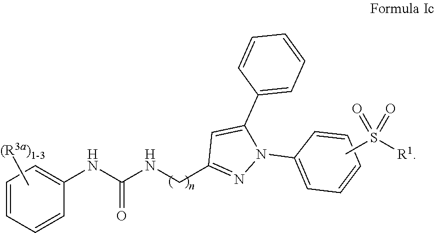

In some embodiments provided herein is a compound of Formula I, wherein R¹ is C₁₋₆ alkyl or —NR^{1a}R^{1b}; Ria and R^{1b} are each independently H and C₁₋₆alkyl; R² is aryl, optionally substituted with C₁₋₆ alkyl; and R³ is cycloalkyl or aryl, each optionally substituted with from 1 to 3 R^{3a} groups wherein each R^{3a} is independently C₁₋₆ alkyl, halogen, C₁₋₆ haloalkyl and C₁₋₆ haloalkoxy. In some other embodiments, R¹ is methyl, ethyl, propyl, —NH₂ and —NMe₂; R² is phenyl, optionally substituted with a member selected from methyl, ethyl or propyl; and R³ is selected from cyclohexyl, cycloheptyl, cyclooctyl, adamantyl or phenyl, wherein the phenyl is optionally substituted with from 1 to 3 R^{3a} groups wherein each R^{3a} is independently methyl, ethyl, propyl, Cl, Br, I, —CF₃ or —OCF₃.

In some embodiments of the compounds of Formula I or Ia, R³ is phenyl optionally substituted with 1 to 3 R^{3a} groups wherein each R^{3a} is independently selected from the group consisting of C₁₋₆ alkyl, C₁₋₆ alkoxy, halogen, C₁₋₆ haloalkyl, and C₁₋₆ haloalkoxy. In some embodiments of the compounds of Formula I or Ia, R³ is phenyl optionally substituted with 1 R^{3a} group wherein R^{3a} is selected from the group consisting of C₁₋₆ alkyl, C₁₋₆ alkoxy, halogen, C₁₋₆ haloalkyl, and C₁₋₆ haloalkoxy. In some embodiments of the compounds of Formula I or Ia, R³ is

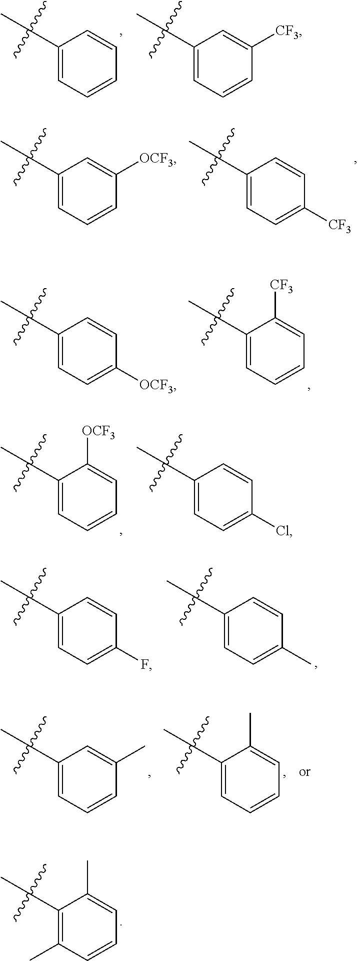

In some embodiments of the compounds of Formula I or Ia. In some embodiments of the compounds of Formula I or Ia, R³ is

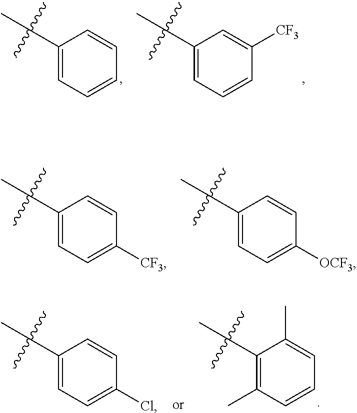

In some embodiments of the compounds of Formula I or Ia, R³ is cycloalkyl optionally substituted with 1 to 3 R^{3a} groups wherein each R^{3a} is independently selected from the group consisting of C₁₋₆ alkyl, C₁₋₆ alkoxy, halogen, C₁₋₆ haloalkyl and C₁₋₆ haloalkoxy. In some embodiments of the compounds of Formula I or Ia, R³ is unsubstituted cycloalkyl. In some embodiments of the compounds of Formula I or Ia, R³ is

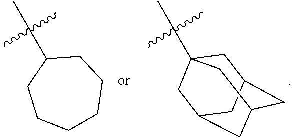 [SMILES: *C1CCCCCC1 ; *C12CC3CC(CC(C3)C1)C2]

In some embodiments of the compounds of Formula I or Ia, R² is phenyl optionally substituted with C₁₋₆ alkyl. In some embodiments of the compounds of Formula I or Ia, R² is unsubstituted phenyl. In some embodiments of the compounds of Formula I or Ia, R¹ is —NR^{1a}R^{1b}. In some embodiments of the compounds of Formula I or Ia, R¹ is —NH₂. In some embodiments of the compounds of Formula I or Ia, R¹ is C₁₋₆ alkyl. In some embodiments of the compounds of Formula I or Ia, R¹ is —CH₃. In some embodiments of the compounds of Formula I or Ia, n is an integer from 1 to 3. In some embodiments of the compounds of Formula I or Ia, n is 1. In some embodiments of the compounds of Formula I or Ia, n is 2. In some embodiments of the compounds of Formula I or Ia, n is 3.

In some other embodiments provided herein is a compound of Formula I, which are dual inhibitors of sEH and COX-2, wherein the compound is selected from:

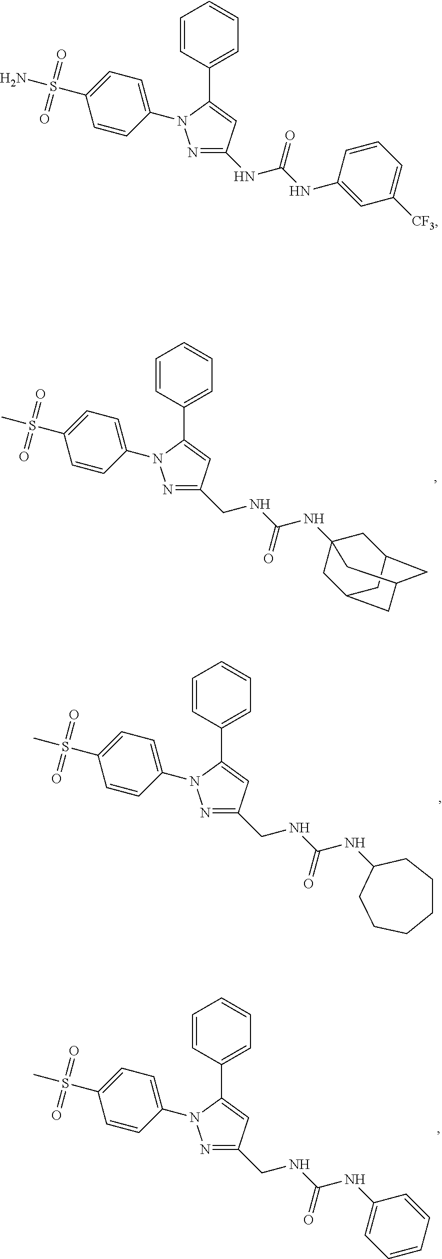 [SMILES: NS(=O)(=O)c1ccc(-n2nc(NC(=O)Nc3cccc(C(F)(F)F)c3)cc2-c2ccccc2)cc1 ; CS(=O)(=O)c1ccc(-n2nc(CNC(=O)NC34CC5CC(CC(C5)C3)C4)cc2-c2ccccc2)cc1 ; CS(=O)(=O)c1ccc(-n2nc(CNC(=O)NC3CCCCCC3)cc2-c2ccccc2)cc1 ; CS(=O)(=O)c1ccc(-n2nc(CNC(=O)Nc3ccccc3)cc2-c2ccccc2)cc1]

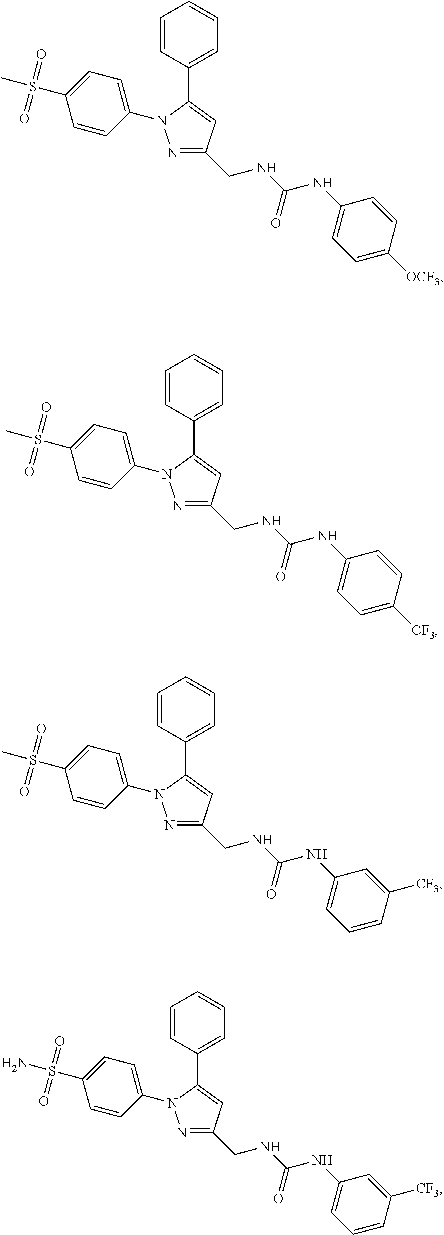 [SMILES: CS(=O)(=O)c1ccc(-n2nc(CNC(=O)Nc3ccc(OC(F)(F)F)cc3)cc2-c2ccccc2)cc1 ; CS(=O)(=O)c1ccc(-n2nc(CNC(=O)Nc3ccc(C(F)(F)F)cc3)cc2-c2ccccc2)cc1 ; CS(=O)(=O)c1ccc(-n2nc(CNC(=O)Nc3cccc(C(F)(F)F)c3)cc2-c2ccccc2)cc1 ; NS(=O)(=O)c1ccc(-n2nc(CNC(=O)Nc3cccc(C(F)(F)F)c3)cc2-c2ccccc2)cc1]

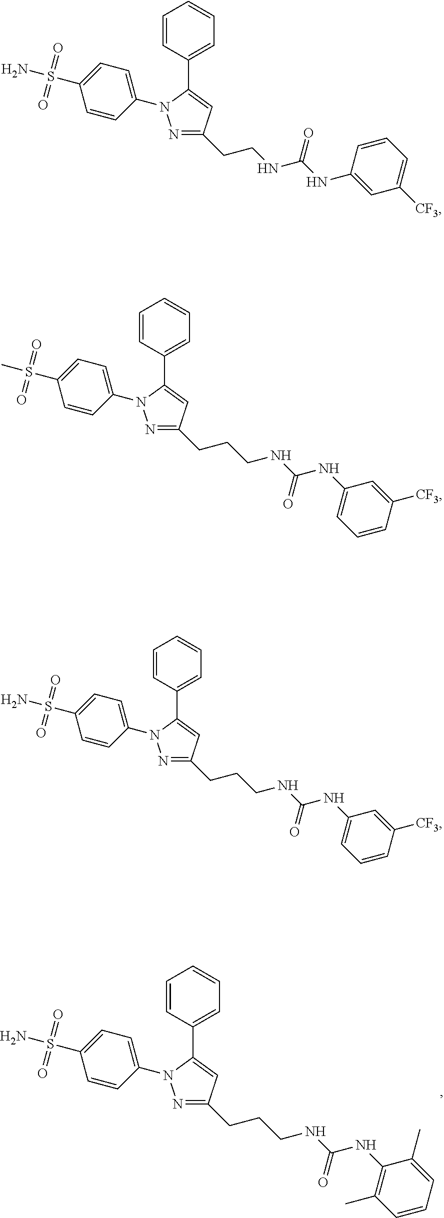 [SMILES: NS(=O)(=O)c1ccc(-n2nc(CCNC(=O)Nc3cccc(C(F)(F)F)c3)cc2-c2ccccc2)cc1 ; CS(=O)(=O)c1ccc(-n2nc(CCCNC(=O)Nc3cccc(C(F)(F)F)c3)cc2-c2ccccc2)cc1 ; NS(=O)(=O)c1ccc(-n2nc(CCCNC(=O)Nc3cccc(C(F)(F)F)c3)cc2-c2ccccc2)cc1 ; Cc1cccc(C)c1NC(=O)NCCCc1cc(-c2ccccc2)n(-c2ccc(S(N)(=O)=O)cc2)n1]

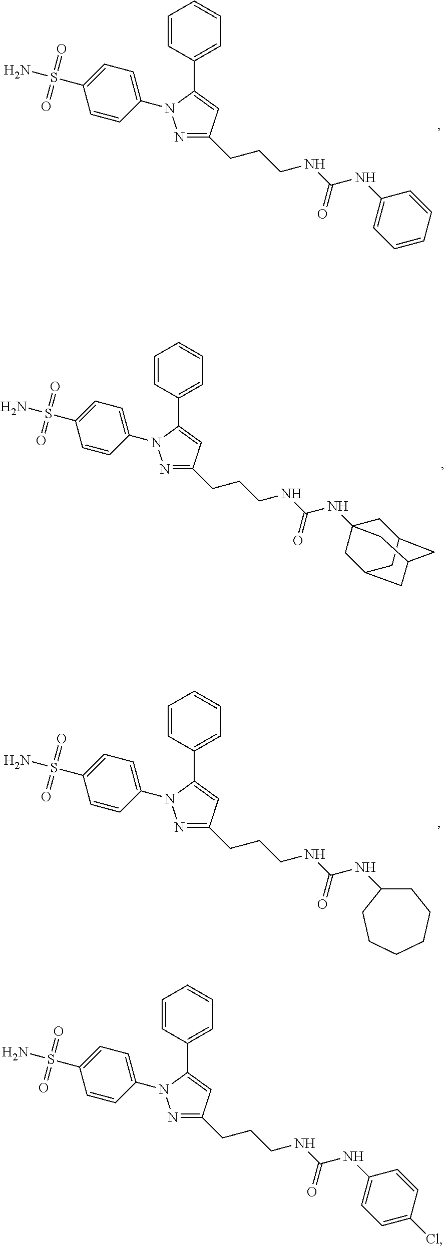 [SMILES: NS(=O)(=O)c1ccc(-n2nc(CCCNC(=O)Nc3ccccc3)cc2-c2ccccc2)cc1 ; NS(=O)(=O)c1ccc(-n2nc(CCCNC(=O)NC34CC5CC(CC(C5)C3)C4)cc2-c2ccccc2)cc1 ; NS(=O)(=O)c1ccc(-n2nc(CCCNC(=O)NC3CCCCCC3)cc2-c2ccccc2)cc1 ; NS(=O)(=O)c1ccc(-n2nc(CCCNC(=O)Nc3ccc(Cl)cc3)cc2-c2ccccc2)cc1]

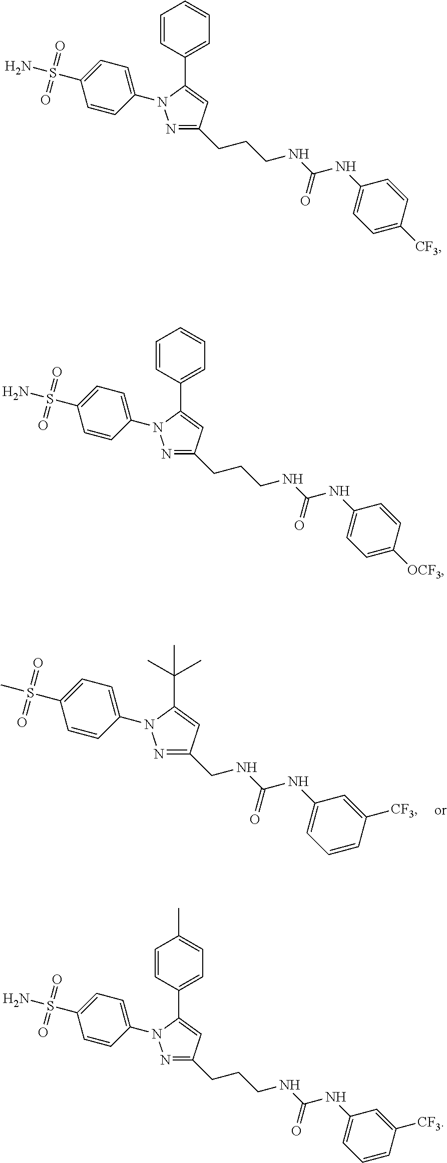 [SMILES: NS(=O)(=O)c1ccc(-n2nc(CCCNC(=O)Nc3ccc(C(F)(F)F)cc3)cc2-c2ccccc2)cc1 ; NS(=O)(=O)c1ccc(-n2nc(CCCNC(=O)Nc3ccc(OC(F)(F)F)cc3)cc2-c2ccccc2)cc1 ; CC(C)(C)c1cc(CNC(=O)Nc2cccc(C(F)(F)F)c2)nn1-c1ccc(S(C)(=O)=O)cc1 ; Cc1ccc(-c2cc(CCCNC(=O)Nc3cccc(C(F)(F)F)c3)nn2-c2ccc(S(N)(=O)=O)cc2)cc1]

In other embodiments, the compound can be:

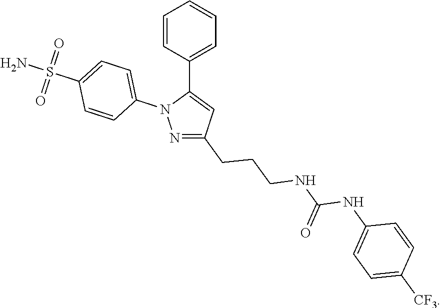 [SMILES: NS(=O)(=O)c1ccc(-n2nc(CCCNC(=O)Nc3ccc(C(F)(F)F)cc3)cc2-c2ccccc2)cc1]

The compounds of the present invention may exist as salts. The present invention includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures), succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present invention contain both basic and acidic functionalities that allow the compounds to be converted into either base or acid addition salts.

Other salts include acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques.

Isomers include compounds having the same number and kind of atoms, and hence the same molecular weight, but differing in respect to the structural arrangement or configuration of the atoms.

It will be apparent to one skilled in the art that certain compounds of this invention may exist in tautomeric forms, all such tautomeric forms of the compounds being within the scope of the invention. Tautomer includes one of two or more structural isomers which exist in equilibrium and which are readily converted from one isomeric form to another.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, the compounds of the present invention may also contain unnatural proportions of atomic isotopes at one or more of the atoms that constitute such compounds. For example, the compounds of the present invention may be radiolabeled with radioactive isotopes, such as for example deuterium (²H), tritium (³H), iodine-125 (¹²⁵I), carbon-13 (¹³C), or carbon-14 (¹⁴C). All isotopic variations of the compounds of the present invention, whether radioactive or not, are encompassed within the scope of the present invention.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

### Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compound as disclosed herein may be derived from reactions and the reactions may be modified by the use of appropriate reagents and conditions, for the introduction of the various moieties found in the formulae as provided herein.

### Pharmaceutical Composition

In some embodiments provided herein is a pharmaceutical composition including a pharmaceutically acceptable excipient and the compound of formula I.

The compounds described herein can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The compounds described herein can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds described herein can be administered transdermally. The compounds described herein can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and either a compound of Formula (I), or a pharmaceutically acceptable salt of a compound of Formula (I).

For preparing pharmaceutical compositions from the compounds described herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the compounds described herein can also be used orally using, for example, push-fit capsules made of gelatin, as well as soft, sealed capsules made of gelatin and a coating such as glycerol or sorbitol. Push-fit capsules can contain the compounds described herein mixed with a filler or binders such as lactose or starches, lubricants such as talc or magnesium stearate, and, optionally, stabilizers. In soft capsules, the compounds described herein may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending a compounds described herein in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the compounds described herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compounds described herein can be delivered by transdermally, by a topical route, formulated as applicator sticks, solutions, suspensions, emulsions, gels, creams, ointments, pastes, jellies, paints, powders, and aerosols.

The compounds and compositions described herein can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The pharmaceutical formulations of the compounds described herein can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms.

In another embodiment, the compounds described herein can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compounds described herein into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, the compound administered and disease or condition treated.

Single or multiple administrations of the compounds described herein can be administered depending on the dosage and frequency as required and tolerated by the patient. The formulations should provide a sufficient quantity of active agent to effectively treat the disease state. Thus, in one embodiment, the pharmaceutical formulations for oral administration of the compounds described herein is in a daily amount of between about 0.5 to about 20 mg per kilogram of body weight per day. In an alternative embodiment, dosages are from about 1 mg to about 4 mg per kg of body weight per patient per day are used. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing parenterally administrable compositions of the compounds described herein will be known or apparent to those skilled in the art and are described in more detail in such publications as Remington's, supra. See also Nieman, In “Receptor Mediated Antisteroid Action,” Agarwal, et al., eds., De Gruyter, New York (1987).

The compounds described herein can be used in combination with one another, with other active agents known to be suitable for use therewith, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

After a pharmaceutical composition including a compound described herein has been formulated in an acceptable carrier, it can be placed in an appropriate container and labeled for treatment of an indicated condition. For administration of the compounds described herein, such labeling would include, e.g., instructions concerning the amount, frequency and method of administration.

In another embodiment, the compositions described herein are useful for parenteral administration, such as intravenous (IV) administration or administration into a body cavity or lumen of an organ. The formulations for administration will commonly comprise a solution of the compositions described herein dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions described herein in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

### Method of Inhibiting sEH and COX

In some embodiments, the disclosure described herein provides a method for inhibiting a soluble epoxide hydrolase, the method including contacting the sEH with an amount of a compound of Formula I sufficient to inhibit the sEH, thereby inhibiting the sEH. In some embodiments, the amount of a compound of Formula I is a therapeutically effective amount. In some other embodiments, the compound further inhibits an enzyme. In other embodiments, the enzyme is a cyclooxygenase enzyme selected from cyclooxygenase-1 (COX-1) or cyclooxygenase-2 (COX-2).

A compound of the present invention inhibits sEH when the concentration of the compound is about 10,000 nM or less and the activity of sEH is reduced by at least 50%. In some embodiments, the concentration of the compound is about 5.00 nM or less; 2,500 nM or less; 1,125 nM or less; 500 nM or less; 250 nM or less; 125 nM or less; 75 nM or less; 30 nM or less; 25 nM or less; 10 nM or less; 5 nM or less; 1 nM or less; 0.5 nM or less; or 0.2 nM or less, and the activity of sEH is reduced by at least 50%. In some other embodiments, the activity of sEH is reduced by at least about 50, 55, 60, 65, 70, 75, 80, 85, 90, 95, 96, 97, 98 or 99%.

A compound of the present invention inhibits COX-2 when the concentration of the compound is about 100 μM or less and the activity of COX-2 is reduced by at least 50%. In some embodiments, the concentration of the compound is about 50 μM or less; 25 μM or less; 13 μM or less; 10 μM or less; 5 μM or less; 2 μM or less; 1 μM or less; 0.5 μM or less; or 0.2 μM or less, and the activity of COX-2 is reduced by at least 50%. In some other embodiments, the activity of COX-2 is reduced by at least about 50, 55, 60, 65, 70, 75, 80, 85, 90, 95, 96, 97, 98 or 99%.

In some other embodiments, the sEH is inhibited without substantially inhibiting a cyclooxygenase enzyme selected from the group consisting of cyclooxygenase-1 (COX-1) and cyclooxygenase-2 (COX-2). In some embodiments, the sEH is inhibited without substantially inhibiting COX-1. For example, COX-1 is considered not substantially inhibited when, in the presence of the composition of the present invention, the activity of COX-1 is not reduced by more than about 50%, relative to the COX-1 activity in the absence of the composition of the present invention. In some embodiments, the activity of COX-1 is not reduced by more than about 40, 35, 30, 25, 20, 15, 10, 5, 2, 1, or 0.5%, relative to the COX-1 activity in the absence of the composition of the present invention.

In some other embodiments, the present invention provides a method for inhibiting a cyclooxygenase enzyme selected from cyclooxygenase-1 (COX-1) or cyclooxygenase-2 (COX-2), the method including contacting a cyclooxygenase enzyme with a compound of Formula I in an amount sufficient to inhibit cyclooxygenase, thereby inhibiting the cyclooxygenase enzyme. In some embodiments, the cyclooxygenase enzyme is COX-2. In some other embodiments, the amount is a therapeutically effective amount. In some embodiments, the COX-2 is inhibited without substantially inhibiting COX-1.

In some embodiments, the present invention provides a method for monitoring the activity of a soluble epoxide hydrolase, the method including contacting the soluble epoxide hydrolase with an amount of a compound of Formula I sufficient to produce a detectable change in the fluorescence of the soluble epoxide hydrolase by interacting with one or more tryptophan residues present in the catalytic site of said soluble epoxide hydrolase, thereby monitoring the activity of the soluble epoxide hydrolase.

The compounds provided herein can be assayed with respect to their ability to inhibit sEH. Additionally, the present invention provides assays and associated methods for monitoring soluble epoxide hydrolase activity, particularly the activity that has been modulated by the administration of one or more of the compounds provided above.

In some embodiments, the present invention provides methods for reducing the formation of a biologically active diol produced by the action of a soluble epoxide hydrolase, the method includes contacting the soluble epoxide hydrolase with an amount of a compound of the present invention, sufficient to inhibit the activity of the soluble epoxide hydrolase and reduce the formation of the biologically active diol. In some other embodiments, the present invention provides methods for stabilizing biologically active epoxides in the presence of a soluble epoxide hydrolase, the method including contacting the soluble epoxide hydrolase with an amount of a compound of the present invention, sufficient to inhibit the activity of the soluble epoxide hydrolase and stabilize the biologically active epoxide. The methods can be carried out as part of an in vitro assay or the methods can be carried out in vivo by monitoring blood titers of the respective biologically active epoxide or diol.

Epoxides and diols of some fatty acids are biologically important chemical mediators and are involved in several biological processes. Epoxy lipids are anti-inflammatory and anti-hypertensive. Additionally, the lipids are thought to be metabolized by beta-oxidation, as well as by epoxide hydration. Soluble epoxide hydrolase is considered to be the major enzyme involved in the hydrolytic metabolism of these oxylipins. The compounds of the present invention can inhibit soluble epoxide hydrolase and stabilize the epoxy lipids both in vitro and in vivo. This activity results in a reduction of hypertension in four separate rodent models. Moreover, the inhibitors show a reduction in renal inflammation associated with, and independent of, the hypertensive models.

The present invention also provides methods for monitoring a variety of lipids in both the arachidonate and linoleate cascade simultaneously in order to address the biology of the system. A GLC-MS system or a LC-MS method can be used to monitor over 740 analytes in a highly quantitative fashion in a single injection. The analytes include the regioisomers of the arachidonate epoxides (EETs), the diols (DHETs), as well as other P450 products including HETEs. Characteristic products of the cyclooxygenase, lipoxygenase, and peroxidase pathways in both the arachidonate and linoleate series can also be monitored. Such methods are particularly useful as being predictive of certain disease states. The oxylipins can be monitored in mammals following the administration of inhibitors of epoxide hydrolase. Generally, EH inhibitors increase epoxy lipid concentrations at the expense of diol concentrations in body fluids and tissues.

sEH inhibition IC₅₀ values for the compounds presented herein can be assayed by a recombinant affinity assay using purified sEHs, from human, mouse or rat, in a fluorescent-based assay. In such an assay, enzymes, e.g., ˜1 nM human sEH, are incubated with a potential inhibitors for 5 min in 25 mM Bis-Tris/HCl buffer (200 μL; pH 7.0) at 30° C. before a substrate, e.g., cyano(2-methoxynaphthalen-6-yl)methyl trans-(3-phenyl-oxyran-2-yl)methyl carbonate, (CMNPC) is added ([S]_{final}=5 μM). Activity is assessed by measuring the appearance of the fluorescent 6-methoxynaphthaldehyde product (λ_{cm}=330 nm, λₑₓ=465 nm) at 30° C. during a 10 min incubation (Spectramax M2; Molecular Device, Inc., Sunnyvale, Calif.). The IC₅₀ values represent the concentration of the inhibitors which reduces the activity by 50%.

A COX Fluorescent Inhibitor Screening Assay Kit (catalog number 700100, Cayman Chemical, Ann Arbor, Mich.) is useful for assaying the ability of the compounds provided herein to inhibit ovine COX-1 and human recombinant COX-2 (% inhibition at 100 μM and IC₅₀ values (M), respectively). Stock solutions of test compounds are dissolved in a minimum volume of DMSO. 10 μl of various concentrations of the test compound solutions, e.g., [I]_{final} between 0.01 and 100 μM, are added to a series of supplied reaction buffer solutions (150 μl, 100 mM Tris-HCl, pH 8.0) with either COX-1 or COX-2 (10 μl) enzyme in the presence of Heme (10 μl) and a fluorometric substrate (10 μl). The reactions are initiated by quickly adding 10 μl of arachidonic acid solution and then incubating the system for two minutes at room temperature. Fluorescence of resorufin, which is produced by the reaction between PGG₂ and fluorometric substrate, ADHP (10-acetyl-3,7-dihydroxyphenoxazine), is analyzed with an excitation wavelength of 535 nm and an emission wavelength of 590 nm. The intensity of this fluorescence is proportional to the amount of resorufin, which is proportional to the amount of PGG₂ present in the well during the incubation. Percent inhibition is calculated by comparison from the 100% initial activity sample value (no inhibitor).

### Method of Inhibiting Fibrotic Progression

In some embodiments, the present invention provides a method of treating or preventing fibrotic progression. In some embodiment, the present invention provides a model of treating or preventing fibrotic progression as described in Example 1.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

The section headings used herein are for organizational purposes only and are not to be construed as limiting the subject matter described.

The invention is further described in the following Examples, which do not limit the scope of the invention described in the claims.

### EXAMPLES

### Example 1: Prophylactic Treatment of IPF Using a Dual Inhibitor of sEH and Cyclooxyuenase-2 (sEH/COX-2)

4-(5-phenyl-3-{3-[3-(4-trifluoromethyl-phenyl)-ureido]-propyl}-pyrazol-1-yl)-benzenesulfonamide (PTUPB) [termed OX1] was compared to Pirfenidone, Nintedanib, and GSK225629415 [termed OX3] for treatment of IPF using an established model for IPF treating, which is prophylactic treatment of mice exposed to the chemotherapeutic agent bleomycin. OX1 has an IC₅₀-value of 2.0 nM for sEH and 100 nM for COX-2. OX3 has an IC₅₀ value of 27 pM against human sEH and >1000 nM for COX-2. Therefore, OX3 is roughly 75-fold more potent against sEH and at least 10 times less potent on COX-2 in vitro than OX1. Pirfenidone and Nintedanib are standard of care for the treatment of idiopathic pulmonary fibrosis and served as reference compounds in the experiment.

C57B/L6 mice were used in the experiments. When used, Bleomycin was administered intra-oropharyngeally, 1.5 U/kg, QD per established protocol. The test articles were dosed via oral gavage in 200 μL/day vehicle. The doses and formulations of test articles were selected based on the literature describing the lowest doses capable of eliciting pharmacological action. There were 6 groups of mice:
p1—No Bleomycin (n=5), vehicle, QD
p2—Bleomycin only (n=10), Bleomycin and vehicle, QD
p3—Bleomycin+OX1 (n=10), Bleomycin and 30 mg/kg of OX1, QD
p4—Bleomycin+OX3 (n=10), Bleomycin and 30 mg/kg of OX3, QD
p5—Bleomycin+Pirfenidone (n=10), Bleomycin and 100 mg/kg of Pirfenidone, BID
p6—Bleomycin+Nintedanib, n=10, Bleomycin and 30 mg/kg of Nintedanib, QD

Animals were dosed with the test articles (or vehicle) starting at day −1 to day 20. On day 1, daily doses of Bleomycin commenced in all but group 1 (negative control) which received the vehicle. On day 21, all animals were weighed and sacrificed. After lungs and bronco-alveolar lavage fluid (BALF) were harvested, they were prepared according to standard procedures for further analysis and histology. All animals were assessed twice a week for body weight and overall appearance. Efficacy endpoints measured in this study included lung weight, total leukocytes, and histology as determined by Mason's Trichrome stain and scored using a modified Ashcroft score. (Hubner, R. H. et al. Standardized Quantification of Pulmonary Fibrosis in Histological Samples. BioTechniques. 2008, 44(4):507-17.)

The following parameters were measured in bronchoalveolar fluid (BALF): 1) soluble collagen and 2) differential leukocyte counts for different leukocyte populations. In addition, measurements of α-SMA as well as the presence of inflammatory cytokines were included in the protocol (work in progress).

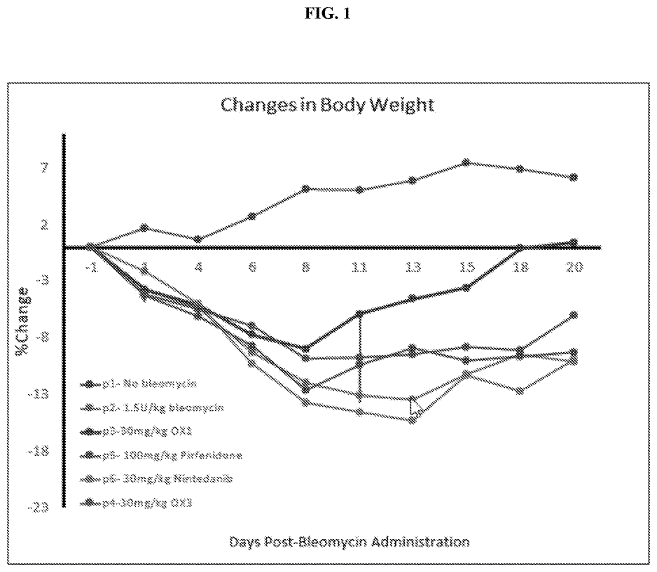

As shown in FIG. 1, mice in all test groups lost weight in days −1 to 8. The OX1 and OX3 groups lost the least weight. In days 8 onward, bleomycin alone, or treatment groups with Pirfenidone, Nintedanib, or OX3 sustained weight loss. During the first week OX1-treated mice followed the same trend, however, these animals then recovered and ended with −1% weight gain compared to day−1. The negative control animals ended the experiment with −6% weight gain compared to day−1.

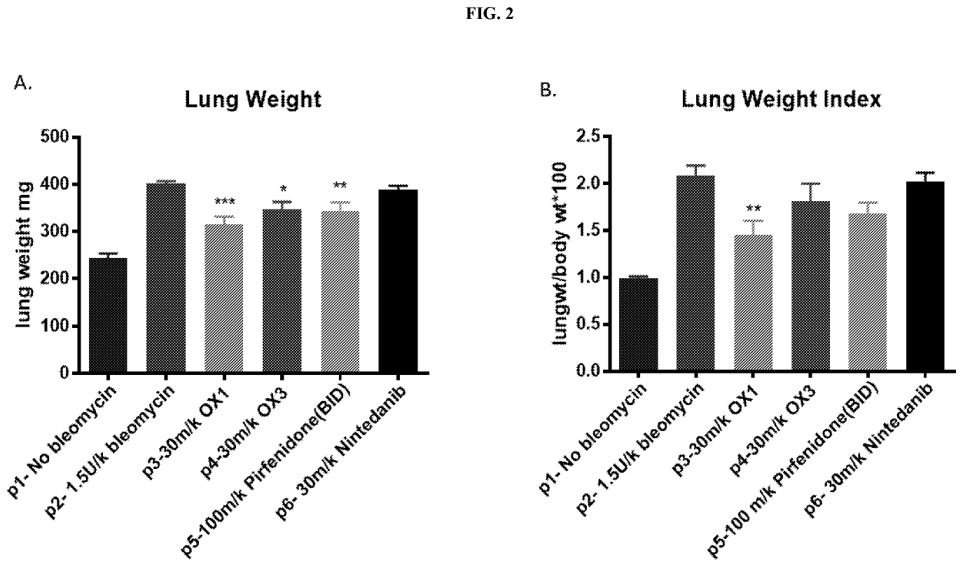

Lung weight data, an indicator of fluid accumulation and inflammation in the lungs, revealed that after Bleomycin treatment OX1 performed best in reducing bronchoalveolar fluid (BALF) accumulation and inflammation (FIG. 2A and FIG. 2B).

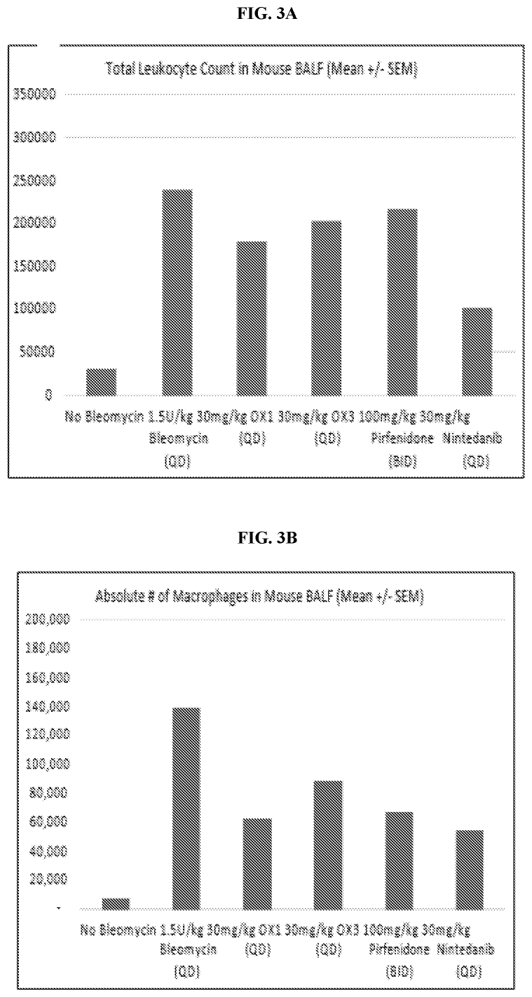

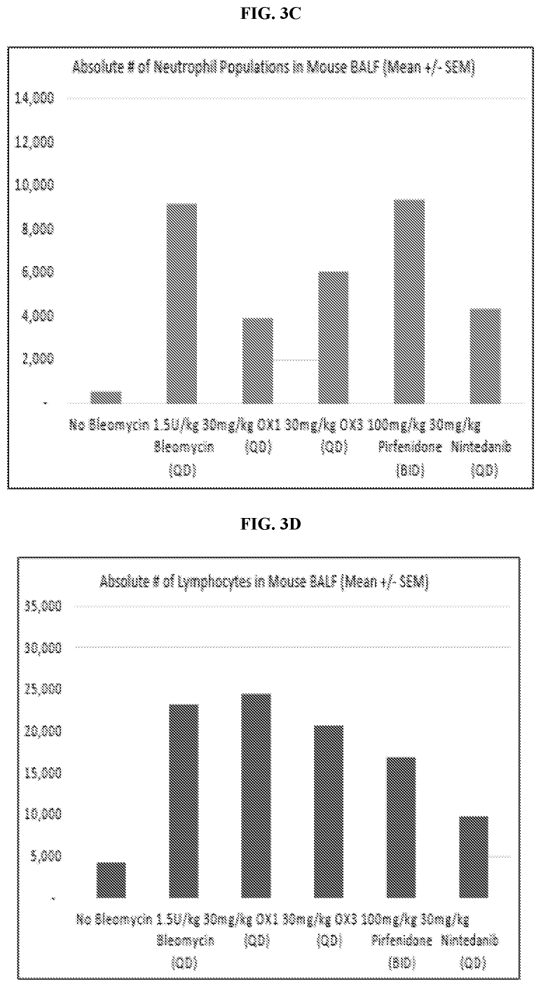

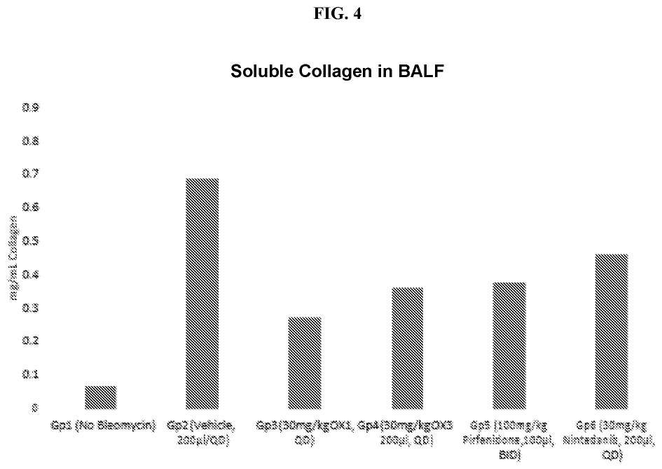

FIG. 3A depicts the Total Cell Count data in BALF. Overall, Nintedanib demonstrated the largest reduction of total number of cells found in BALF. OX1, OX3, and Pirfenidone did not demonstrate the same level of reduction and performed similarly. However, the total leukocyte count does not shed light on the relative make-up of the sub-types which are critical to understanding etiology and potential treatment of lung fibrosis. A deeper analysis of cell types is shown in FIG. 3B-3D. As shown in FIG. 3B, all test articles performed equally well in reducing the number of macrophages in BALF. Although not statistically significant, there were more macrophages in OX3 BALF samples. This is a positive attribute for all these reagents, since macrophages play a key role in homeostatic regulation as well as during development of inflammation and lung fibrosis. These cells and their products are intricately involved in key stages of fibrosis. They are often found in proximity of collagen-producing myofibroblasts and can secrete a number of profibrotic mediators, chemokines, and matrix metalloproteases. During injury, macrophages readily acquire a phenotype which promotes fibroproliferation, required for fibrosis. Their normal role includes collagen uptake or degradation. However, macrophages isolated under pulmonary fibrotic conditions (mice or patients) can secrete neutrophil chemoattractants and exacerbate the condition.

As shown in FIG. 3C, OX1 and Nintedanib were effective in reducing neutrophil migration into BALF, in contrast to Pirfenidone which did not exhibit any effects in this regard. OX3 was somewhat effective. The role of neutrophils in causing tissue damage in the alveoli and etiology of pulmonary lung fibrosis has been well established 19 and a reduction in their levels can ameliorate fibrosis.

FIG. 3D shows the lymphocyte count data. The key lymphocytic infiltration in fibrosis is due to the T lymphocytes. T lymphocytes have a protective role against fibrosis in patients with IPF and scleroderma, as well as in the bleomycin-induced pulmonary fibrosis. As expected, the data shown in FIG. 3D indicates that without Bleomycin challenge one observes the lowest levels of lymphocytes in the BALF. When challenged with Bleomycin, a large number of lymphocytes infiltrate into the BALF as a repair response. OX1 treatment appears to be most effective at maintaining a large number of lymphocytes in place, which might be beneficial in light of the totality of evidence discussed above. In contrast to OX1, the number of lymphocytes in BALF seems to trend increasingly lower with OX3, Pirfenidone, and Nintedanib. This novel finding in part explains the overall treatment superiority of OX1 in treating the bleomycin-treated mice.

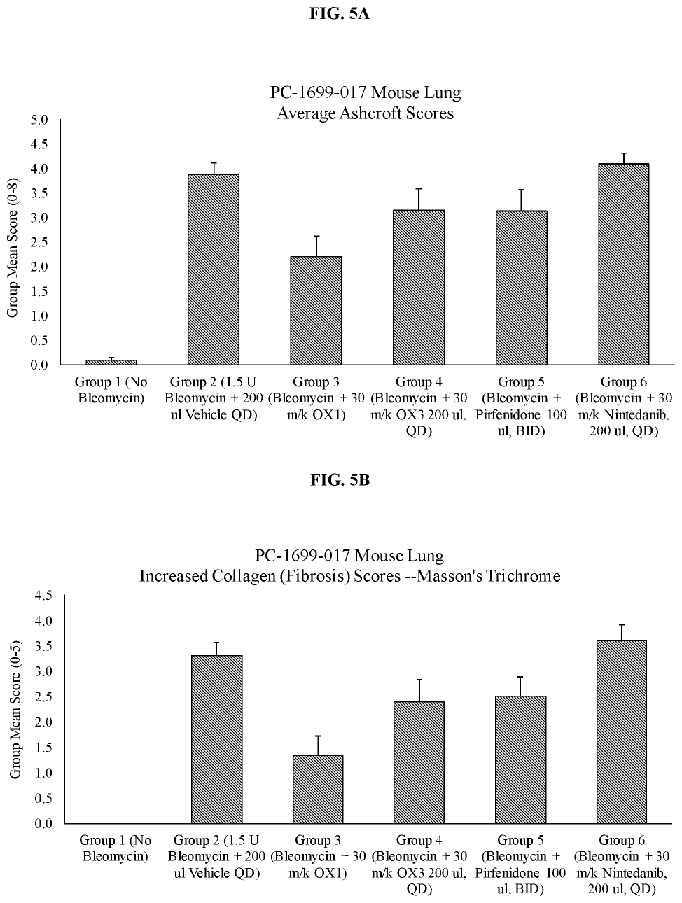

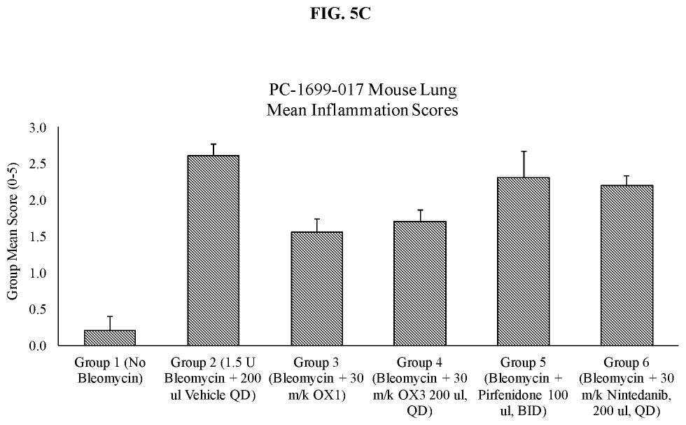

FIG. 4 shows the concentrations of soluble collagen in the BALF of mice treated with different test articles. This is in agreement with histological observations in which the lowest tissue deposition of collagen was observed in OX1-treated animals (FIG. 5B). It can therefore be concluded that OX1 was the most effective in controlling the overall collagen production in the lungs.

After weighing and removal of BALF from the lungs, they were inflated and fixed using 10% neutral buffered formalin for histopathology analysis. Fifty-four formalin-fixed lung samples from mice were submitted to HistoTox Labs and were processed routinely. Two slides from each block were sectioned and stained with hematoxylin and eosin (H&E) or Masson's trichrome. Glass slides were evaluated with light microscopy by a board-certified veterinary pathologist. Lung sections were scored according to the modified Ashcroft scale. Briefly, scores for five representative 200× microscopic fields per sample were averaged to obtain a mean score for each animal.
Grade 0=Normal lung
Grade 1=Minimally detectable thickening of alveolar walls
Grade 2=Mild thickening of alveolar walls
Grade 3=Moderate contiguous thickening of walls with fibrous nodules
Grade 4=Thickened septae and confluent fibrotic masses totaling less than 10% of the microscopic field
Grade 5=Increased fibrosis with definite damage to lung structure and formation of fibrous bands or small fibrous masses between 10-50% of the microscopic field
Grade 6=Large contiguous fibrotic masses consolidating more than 50% of the microscopic field
Grade 7=Severe distortion of structure and large fibrous areas
Grade 8=Total fibrous obliteration of lung within the microscopic field
   Additional lesions in H&E- and trichrome-stained lung sections were graded for severity 0-5 (0=not present/normal, 1=minimal, 2=mild, 3=moderate, 4=marked, 5=severe).

Animals administered bleomycin developed expected lesions consistent with pulmonary fibrosis, consisting of expansion of alveolar walls, formation of nodular masses and partial obliteration of lung architecture by variably dense fibrous connective tissue (collagen). Collagen identified with Masson's trichrome staining ranged from thin, pale blue fibers to dense, intense blue bands of fibrous tissue. In areas of fibrosis, alveoli were occasionally lined by hypertrophied and hyperplastic type II pneumocytes, and contained foamy alveolar macrophages. Infiltration and aggregation of mixed inflammatory cells (lymphocytes, plasma cells and occasional macrophages and neutrophils) in peribronchiolar and perivascular zones was present, with variable extension into areas of fibrosis. Subacute bronchiolar inflammation and intra-airway plant matter, consistent with aspiration pneumonia was sporadically present, and was characterized by infiltration and aggregation of neutrophils, macrophages and multinucleated giant cells surrounding plant material and keratin, variably organized into the bronchiolar wall by fibrous tissue. In one sample (Group 3, Animal 774), abscess formation was present. Lung lobes containing aspiration lesions were not considered for Ashcroft or fibrosis scoring. Lesions of fibrosis and inflammation were absent in mice that did not receive bleomycin (Group 1).

A summary of histopathology data is presented in FIG. 5A-5C. FIG. 5A and FIG. 5B represent two different methods used to investigate changes in lung architecture and collagen deposition as a result of fibrosis. In both cases, OX1 performs better than OX3 and the two marketed drugs. Mean Ashcroft scores (FIG. 5A) and increased collagen (fibrosis) scores (trichrome; FIG. 5B) were the highest in vehicle (Group 2) and Nintedanib (Group 6) treated animals. Mean Ashcroft and fibrosis scores were markedly reduced in OX1-treated animals (Group 3) compared to vehicle-treated (Group 2). Treatment with OX3 (Group 4) and Pirfenidone (Group 5) was also associated with slightly lower Ashcroft and fibrosis scores compared to vehicle treatment (Group 2). FIG. 5C shows the inflammatory scores given to each group of animals based on observed infiltrating inflammatory cells on the slides. Mean scores for mixed peribronchiolar/perivascular inflammation were similar in vehicle (Group 2), Pirfenidone (Group 5) and Nintedanib (Group 6) treated mice (FIG. 3), while scores were lower in animals treated with OX1 (Group 3) or OX3 (Group 4). Both OX1 and OX3 outperformed Pirfenidone, and Nintedanib. Without being bound by theory, this is believed to be due to the anti-inflammatory nature of sEH inhibition by these compounds.

According to the data shown above, we have shown effective treatment of IPF with sEH inhibitors OX1 and OX3. OX1 and OX3 were as effective, or more effective, against IPF than Pirfenidone or Nintedanib. In particular, the dual inhibitor of sEH and COX-2, OX1, was more effective than the mono inhibitor of sEH, OX3, and more effective than Pirfenidone or Nintedanib.

### Example 2: Therapeutic Effects of OX1 in a Bleomycin-Induced Idiopathic Pulmonary Fibrosis Mouse Model

The purpose of this study is to induce fibrotic lung injury in mice via a single administration of a glycopeptide antibiotic, bleomycin, in order to evaluate the efficacy of dual sEH and COX-2 inhibitor OX-1 in treating and reducing the symptoms associated with idiopathic pulmonary fibrosis. Pirfenidone, standard of care for the treatment of idiopathic pulmonary fibrosis, was used as a reference compound.

Male C57BL/6 mice from Charles River Laboratories aging of 7-8 weeks and weighing around 18-22 g at the time of their enrollment were used in the study.

Doses of OX1 at 60 mg/kg, Pirfenidone at 100 mg/kg, and the disease inducer Bleomycin at 1.5 mg/kg were used. A dosing rate of 10 mL/kg (approximately 0.2 mL/mouse according to their body weight) of OX1 was administered orally once daily to the mice. The mice received OX1 at 60 mg/kg once daily. A dosing rate of 10 mL/kg (approximately 0.2 mL/mouse according to their body weight) of Pirfenidone solution was administered once daily to the mice of the groups receiving Pirfenidone orally at the dose of 100 mg/kg once daily. A single intratracheal instillation of approximately 50 μL (1.5 mg/kg with a dosing rate of 2.5 mL/kg in sterile 0.9% saline) of bleomycin solution was administered to all mice except the sham group on day 0. Animals in the Sham group received one intratracheal instillation of approximately 50 μL of 0.9% saline solution on Day 0 and returned to their respective cages.

TABLE 1

| | | Bleo- | | Route | Drug | | |
|---|---|---|---|---|---|---|---|
| | | mycin | | of | Treat- | Har- | Gr. |
| Gr. | Group | Instil- | Treatment | Admin- | ment | vest | Size |
| # | Description | lation | dose | istration | Duration | Day | (n=) |
| 1 | Sham | No | 10 ml/kg | Oral | Day 7 | Day | 6 |
| | (Vehicle) | (0.9% | Once daily | Gavage | to 21 | 21 | |
| | | saline) | | | | | |
| 2 | Negative | Yes | 10 mL/kg | Oral | Day 7 | Day | 10 |
| | Control | (Day 0) | Once daily | Gavage | to 21 | 21 | |
| | (Vehicle) | | | | | | |
| 3 | OX1 | Yes | 60 mg/kg | Oral | Day 7 | Day | 10 |
| | | (Day 0) | Once daily | Gavage | to 21 | 21 | |
| 4 | Pirfenidone | Yes | 100 mg/kg | Oral | Day 7 | Day | 10 |
| | | (Day 0) | Once daily | Gavage | to 21 | 21 | |

Treatment with the vehicle or test articles were initiated and administered starting on day 7 to day 21 as scheduled and described in Table 1 above. All test articles, reference compound and vehicle were administered to animals orally once daily. Food and water were given ad libitum. Daily observations of the behavioral and general health status of the animals were done. Daily body weight was also measured.

Prior to physiological assessment, all mice were introduced to the plethysmograph chamber environment. After the acclimatization period, the functional respiratory parameters were assessed by the whole-body plethysmograph on Day 0 (before bleomycin instillation), 7, 14 and 21 post-bleomycin instillation. Each measurement was performed with a mouse placed alone in an unrestrained whole-body plethysmography chamber (Buxco Electronics Inc.) to measure respiratory functions. The functional respiratory parameters analyzed included; the respiratory rate, the PenH (pulmonary congestion index) and the inspiratory/expiratory time measurements.

On the surgery day (Day 21), the mice were anesthetized, instrumented and arterial blood oxygen saturation (SpO2) was recorded. Blood oxygen saturation was read off of the pulse oximeter (STARR Life Sciences MouseOx Plus system, Oakmont, Pa.) with the probe attached to the right thigh of the animal. The saturation values were measured in percentage (%). At the moment of the sacrifice, the mouse thoracic cavity was open to expose the lungs and all mice were euthanatized by exsanguination. The trachea was then connected to the cannula of a perfusion system. The left lung was clamped while 1.5 mL of cold PBS solution (3×500 μL) was injected by the trachea to perform a bronchoalveolar lavage fluid (BALF) on the right lobe of the lungs and was collected on ice for further analysis. The total cells count with cells differential, the cytokine levels including TNF-α, TGF-β, IL-4, IL-5 and IL-17a, the total protein content as well as the soluble collagen were assessed in the BALF samples. Once collected, 300 μL of the BALF was transferred in an EDTA tube, kept at 2-4° C. and shipped to Biovet Inc. for the total cells count with cells differential analysis. The remaining BALF sample was centrifuged at 1200 rpm for 10 minutes at 4° C. to remove cells. The supernatant was aliquots and frozen at−80° C. until analysis was performed. The BALF cytokine levels were analyzed using a Luminex assay (Eve Technologies) and the determination of the total protein content (Bradford assay) as well as the soluble collagen assessment (Sircol assay) in BALF were done.

For each mouse, the lungs were harvested, weighed, flushed with 0.9% NaCl and inflated with 10% neutral buffered formalin solution (NBF). The left lobe was kept in fixative and the lower third of the left lobe tissue was sent for the histopathological analysis.

Results are shown as means±SEM. Comparisons were made on normally distributed data using ANOVA, followed by a Fisher post hoc test to assess the difference between BLM+vehicle group with Graph Pad Prism Software version 8.0 (San Diego, Calif., USA). Differences were considered statistically significant when P values were less than 0.05. * Shown difference versus Sham animals and † shown difference versus BLM+Vehicle group. * Means P<0.05, ** P<0.01, *** P<0.001 while † means P<0.05, †† P<0.01 and ††† P<0.001.

### Results

When OX1 at 60 mg/kg is compared to the vehicle treated mice, it can be observed that this treatment alleviated the body weight loss induced by the bleomycin. The survival rates of animals were increased from 66.7% in the vehicle BLM-treated mice to 77.8 in the OX1 treated groups (Table 2).

TABLE 2

| | | Body weight | Body weight | Percent |
|---|---|---|---|---|
| | Statistical | changes from | changes from | Survival |
| Treatment | parameters | day 0 (%) | day 7 (%) | (%) |
| Sham | Mean | 12.69 | 5.46 | 100.00 |
| | SEM | 2.61 | 1.10 | n/a |
| | n | 8 | 8 | 8 |
| BLM + | Mean | 3.73 * | 6.80 | 66.7 |
| Vehicle | SEM | 3.61 | 3.32 | n/a |
| (10 ml/kg) | n | 8 | 8 | 8 |
| BLM + | Mean | 7.89 | 11.63 | 77.8 |
| OX1 | SEM | 1.04 | 2.27 | n/a |
| (60 mg/kg) | n | 7 | 7 | 7 |
| BLM + | Mean | 2.47 | 6.49 | 91.70 |
| Pirfenidone | SEM | 1.41 | 0.97 | n/a |
| (100 mg/kg) | n | 11 | 11 | 11 |

Lung inflammation was evaluated by measuring the amount of infiltrating immune cells found in the bronchoalveolar lavage fluid (Table 3). The treatment with OX1 reduced the total cell count compared to the vehicle BLM-treated mice. Although not significant, the cell count decreases were associated with a reduction of the lymphocyte as well as the neutrophil cells number, when compared to the vehicle treated group.

TABLE 3

| | | Total cell | | | | |
|---|---|---|---|---|---|---|
| | Statistical | counts | Macrophages | Lymphocytes | Neutrophils | Eosinophils |
| Treatment | parameters | (cells) | (cells) | (cells) | (cells) | (cells) |
| Sham | Mean | 111456 | 96093 | 14686 | 480 | 197 |
| | SEM | 13832 | 11505 | 10589 | 338 | 197 |
| | n | 8 | 8 | 8 | 8 | 8 |
| BLM + | Mean | 344472*** | 204635** | 130527*** | 9310* | 0 |
| Vehicle | SEM | 37372 | 19738 | 25762 | 2636 | 0 |
| (10 ml/kg) | n | 17 | 17 | 17 | 17 | 17 |
| BLM + | Mean | 279643 | 204879 | 71346 | 2837 | 580 |
| OX1 (60 | SEM | 42265 | 25731 | 21881 | 2014 | 580 |
| mg/kg) | n | 7 | 7 | 7 | 7 | 7 |
| BLM + | Mean | 386500 | 204110 | 174517 | 6999 | 874 |
| Pirfenidone | SEM | 57418 | 27611 | 50833 | 2546 | 468 |
| (100 mg/kg) | n | 11 | 11 | 11 | 11 | 11 |

To assess the effect of OX1 on the inflammation implicated in the fibrosis development, the levels of some cytokines/chemokines were quantified in the BALF on day 21 (Table 4A and 4B). OX1 had no effect on TNFα and IL-17 levels, but a few tendencies were observed. OX1 seemed to blunt the increase of MIG, IP-10, and to a greater extent, LIF.

TABLE 4A

| | Statistical | Total BALF cytokines (pg) | | | |
|---|---|---|---|---|---|
| Treatment | parameters | TNFa | IL-4 | IL-5 | IL-17 |
| Sham | Mean | 0.38 | 0.09 | 0.31 | 0.09 |
| | SEM | 0.38 | 0.08 | 0.31 | 0.08 |
| | n | 8 | 8 | 8 | 8 |
| BLM + | Mean | 0.15 | 0.05 | 10.36 | 0.01 |
| Vehicle | SEM | 0.15 | 0.04 | 7.34 | 0.01 |
| (10 ml/kg) | n | 8 | 8 | 8 | 8 |
| BLM + | Mean | 0.00 | 0.09 | 2.49 | 0.02 |
| OX1 | SEM | 0.00 | 0.09 | 1.65 | 0.02 |
| (60 mg/kg) | n | 7 | 7 | 7 | 7 |
| BLM + | Mean | 0.00 | 0.00 | 2.17 | 0.00 |
| Pirfenidone | SEM | 0.00 | 0.00 | 1.13 | 0.00 |
| (100 mg/kg) | n | 11 | 11 | 11 | 11 |

TABLE 4B

| | Statistical | Total BALF cytokines (pg) | | | |
|---|---|---|---|---|---|
| Treatment | parameters | IL-6 | IP-10 | MIG | LIF |
| Sham | Mean | 0.35 | 2.76 | 3.45 | 1.03 |
| | SEM | 0.27 | 0.54 | 0.95 | 0.34 |
| | n | 8 | 8 | 8 | 8 |
| BLM + | Mean | 476.18 | 10.93 ** | 7.93 | 42.34 ** |
| Vehicle | SEM | 469.46 | 3.29 | 1.87 | 29.54 |
| (10 ml/kg) | n | 8 | 8 | 8 | 8 |
| BLM + | Mean | 9.50 | 8.72 | 5.98 | 12.26 |
| OX1 | SEM | 6.05 | 2.36 | 1.46 | 5.73 |
| (60 mg/kg) | n | 7 | 7 | 7 | 7 |
| BLM + | Mean | 11.98 | 15.24 | 8.58 | 24.67 |
| Pirfenidone | SEM | 8.32 | 6.27 | 1.96 | 10.93 |
| (100 mg/kg) | n | 11 | 11 | 11 | 11 |

As an indicator of lung fibrosis development, the total TGFβ and the soluble collagen in BALF were quantified (Table 5). As expected, total TGFβ and the soluble collagen of the vehicle treated group were significantly increased from 70.6 to 347.6 pg and from 19.5 to 118.4 μg, respectively, compared to the sham. The treatment with OX1 reduced the elevation of the TGFβ to 299.3 and blunted the soluble collagen increase to 60.4.

TABLE 5

| | Statistical | Soluble collagen | Total TGFβ |
|---|---|---|---|
| Treatment | parameters | (μg) | (pg) |
| Sham | Mean | 19.52 | 70.59 |
| | SEM | 9.91 | 17.50 |
| | n | 8 | 8 |
| BLM + | Mean | 118.35 ** | 347.64 *** |
| Vehicle | SEM | 34.44 | 88.70 |
| (10 ml/kg) | n | 17 | 8 |
| BLM + | Mean | 60.38 | 299.31 |
| OX1 | SEM | 24.90 | 98.23 |
| (60 mg/kg) | n | 7 | 7 |
| BLM + | Mean | 197.82 | 472.64 |
| Pirfenidone | SEM | 54.87 | 117.22 |
| (100 mg/kg) | n | 11 | 11 |

BLM significantly increased the Ashcroft score to 4.7 compared to the sham group with a score at 0.2 (Table 6). OX1 at 60 mg/kg significantly reduced the Ashcroft score to 3.6 with a similar effect ofPirfenidone treatment at 100 mg/kg. Pulmonary fibrosis is characterized by excessive collagen deposition in the lung with subsequent lung pulmonary structure modifications. BLM significantly increased the collagen deposition and the percentage of pulmonary foci in the lung on day 21, as indicated in Table 6. Although, OX1 at 60 mg/kg did not significantly affect the percentage of collagen content as well as pulmonary foci on day 21, it did show a comparable effect to the Pirfenidone at 100 mg/kg.

TABLE 6

| | Statistical | Ashcroft | Pulmonary | Collagen |
|---|---|---|---|---|
| Treatment | parameters | score | foci (%) | content (%) |
| Sham | Mean | 0.2 | 0.74 | 3.64 |
| | SEM | 0.1 | 0.21 | 0.25 |
| | n | 8 | 8 | 8 |
| BLM + | Mean | 4.7 *** | 9.01 *** | 6.30 *** |
| Vehicle | SEM | 0.3 | 2.43 | 0.58 |
| (10 ml/kg) | n | 17 | 17 | 17 |
| BLM + | Mean | 3.6 † | 6.19 | 6.02 |
| OX1 | SEM | 0.4 | 2.45 | 0.45 |
| (60 mg/kg) | n | 7 | 7 | 7 |
| BLM + | Mean | 3.5 †† | 7.46 | 5.91 |
| Pirfenidone | SEM | 0.4 | 2.24 | 0.50 |
| (100 mg/kg) | n | 11 | 11 | 11 |

### Example 3: Fibroblast-to-Myofibroblast Transition (FMT) Assay

OX1 was evaluated in a primary cell-based Fibroblast-to-Myofibroblast Transition (FMT) assay; an in vitro assay relevant to idiopathic pulmonary fibrosis (TPF). Human primary lung fibroblasts from three (3) IPF donors (IPF06, IPF07, and IPF08) were triggered using 1.25 ng/ml TGF-b1 to induce FMT, one hour after application of compounds. OX1 and Nintedanib (approved drug for TPF) were applied in 0.100 DMSO in semi-log dilutions with the top concentration at 10 mM to construct eight (8)-point concentration response curve (CRC) in biological duplicates (ie. on two (2) different cell plates at on (1) occasion). SB525334 was used at 1 mM in 0.1% DMSO as a positive validation control. The readouts at 72 hours were high content analysis (HCA) for aSMA expression and DAPI staining for nuclear count as measurement of potential cytotoxic effects. As shown in Table 7, OX1 and Nintedanib were similar in their IC₅₀ for inhibiting a-SMA. Dose-response was observed for both compounds in this assay. The cells treated with these compounds showed a tendency for loss of nuclei.

TABLE 7

| | αSMA inhibition IC50 (μM) | | | | | Loss |
|---|---|---|---|---|---|---|
| Compound | Patient Identifier | | | | | of |
| ID | IPF06 | IPF07 | IPF08 | Mean | SD | Nuclei |
| OX1 | 1 | 1.3 | 1 | 1.1 | 0.2 | Yes |
| Nintedanib | 0.6 | 0.6 | 0.4 | 0.5 | 0.1 | Yes |

### Example 4: Synthesis and Testing of Dual Inhibitors of Seh and Cyclooxyuenase-2 (sEH/COX-2)

Dual inhibitors of sEH/COX-2 include, without limitation, those that possess the sEH pharmacophore:

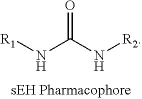

Dual inhibitors of sEH/COX-2 also include, without limitation, those disclosed in U.S. Pat. No. 9,096,532. Exemplary dual inhibitors are synthesized and test for sEH and COX-2 inhibition in vitro. Structure-activity-relationships (SAR) are used to refine compounds to identify progressively more potent dual inhibitors of sEH/COX-2. In addition, physicochemical and ADME attributes are refined to arrive as superior pharmacologically superior compounds. Candidates are further tested in the bleomycin mouse as described in Example 1. Candidates are further tested in clinical trials.

### Example 5: Identification of Secondary Targets (Seh/Cox-2)

Many enzymatic or growth-factor targets other than sEH have been implicated in IPF. Inhibiting these in addition to sEH synergistically treats or prevents fibrotic progression. A review of the RCSB Protein Data Bank showed that several key kinases, enzymes, and growth factors implicated in IPF have ligand-bound structures, such as, without limitation: FLT3, PDGFR-α, PDGFR-β, VEGFR-1, VEGFR-2, COX-2, 5-LOX, FGFR, TGF-β, and CCR1. Inhibitors of these kinases, enzymes, and growth factors are tested in in vitro and in vivo assays known in the art. Inhibitors of these kinases, enzymes, and growth factors are test in a fibroblast assay for fibrotic progression. Inhibitors of these kinases, enzymes, and growth factors are further test in a fibroblast assay for fibrotic progression in the presence of soluble inhibitors of sEH. Based on these results, secondary targets and associated inhibitors are prioritized for synthesis of dual inhibitors by methods known in the art. These dual inhibitors are refined by SAR and tested in the bleomycin mouse and/or in clinical trials as described in Example 4. More specifically, synthesized compounds are screened in vitro in the following or any other sequence:
1) Compounds are first tested in sEH inhibition assays. Those with proper level of sEH inhibition are tested for inhibition of the secondary enzyme.
2) Compounds with proper level of inhibition towards both enzymes are tested in the fibroblast proliferation assay.
3) Compounds deemed active in the proliferation assay are tested in the metabolic stability assays to provide a measure of drug-likeness and fit for in vivo testing. The mouse stability data is geared toward pharmacology studies, while the human microsomal data provides an insight into quality of human PK data.

What is claimed is:

1. A method for treating or preventing a disease associated with fibrotic progression in a subject in need thereof, comprising administering a dual inhibitor of sEH and a secondary target.

2. The method of claim 1, wherein the secondary target is selected from the group consisting of FLT3, PDGFR-α, PDGFR-β, VEGFR-1, VEGFR-2, COX-2, 5-LOX, FGFR, TGF-β, and CCR1.

3. The method of claim 1, wherein the dual inhibitor comprises a sEH pharmacophore selected from the group consisting of Ureas, Carbamates, Amides, and Pyrazoles.

4. The method of claim 3, wherein the sEH pharmacophore comprises the structure:

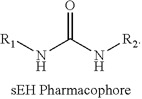

5. The method of claim 1, wherein the dual inhibitor comprises any of the compounds disclosed in U.S. Pat. No. 9,096,532.

6. The method of claim 2, wherein the secondary target is COX-2.

7. The method of claim 6, wherein the dual inhibitor is a compound of Formula I: 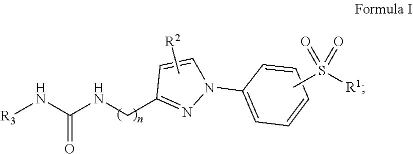
wherein:
R¹ is selected from the group consisting of C₁₋₆ alkyl, —NR^{1a}R^{1b} and cycloalkyl;
R^{1a} and R^{1b} are each independently selected from the group consisting of H and C₁₋₆ alkyl;
R² is selected from the group consisting of C₁₋₆ alkyl, cycloalkyl and aryl, wherein the cycloalkyl and aryl are each optionally substituted with C₁₋₆ alkyl;
R³ is selected from the group consisting of cycloalkyl and aryl, each optionally substituted with from 1 to 3 R^{3a} groups wherein each R^{3a} is independently selected from the group consisting of C₁₋₆ alkyl, C₁₋₆ alkoxy, halogen, C₁₋₆ haloalkyl, and C₁₋₆ haloalkoxy;
subscript n is an integer from 0 to 6;
and salts and optical isomers thereof.

8. The method of claim 7, wherein the dual inhibitor is a compound of Formula Ia:

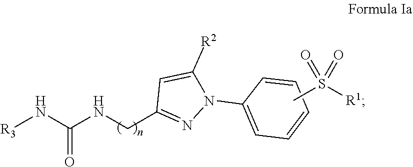

9. The method of claim 7 or 8, wherein R³ is phenyl optionally substituted with 1 to 3 R^{3a} groups wherein each R^{3a} is independently selected from the group consisting of C₁₋₆ alkyl, C₁₋₆ alkoxy, halogen, C₁₋₆ haloalkyl, and C₁₋₆ haloalkoxy.

10. The method of any one of claims 7-9, wherein R³ is phenyl optionally substituted with 1 R^{3a} group wherein R^{3a} is selected from the group consisting of C₁₋₆ alkyl, C₁₋₆ alkoxy, halogen, C₁₋₆ haloalkyl, and C₁₋₆ haloalkoxy.

11. The method of any one of claims 7-10, wherein R³ is

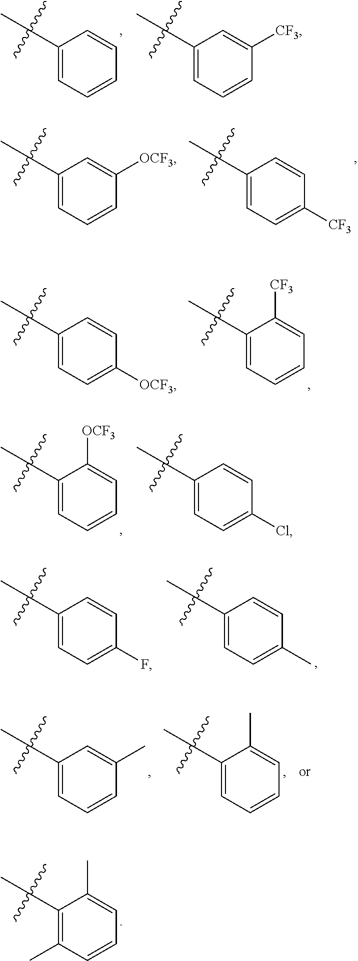

12. The method ofany one of claims 7-10, wherein R³ is

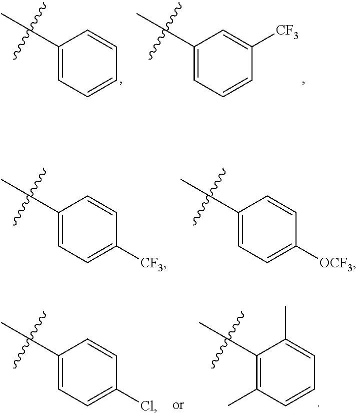

13. The method of claim 7 or 8, wherein R³ is cycloalkyl optionally substituted with 1 to 3 R^{3a} groups wherein each R^{3a} is independently selected from the group consisting of C₁₋₆ alkyl, C₁₋₆ alkoxy, halogen, C₁₋₆ haloalkyl and C₁₋₆ haloalkoxy.

14. The method of claim 13, wherein R³ is unsubstituted cycloalkyl.

15. The method of claim 14, wherein R³ is

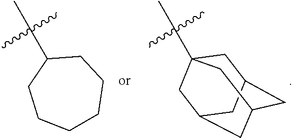 [SMILES: *C1CCCCCC1 ; *C12CC3CC(CC(C3)C1)C2]

16. The method of any one of claims 7-15, wherein R² is phenyl optionally substituted with C₁₋₆ alkyl.

17. The method of any one of claims 7-16, wherein R² is unsubstituted phenyl.

18. The method of any one of claims 7-17, wherein R¹ is —NR^{1a}R^{1b}.

19. The method of any one of claims 7-18, wherein R¹ is —NH₂.

20. The method of any one of claims 7-17, wherein R¹ is C₁₋₆ alkyl.

21. The method of claim 20, wherein R¹ is —CH₃.

22. The method of any one of claims 7-21, wherein n is an integer from 1 to 3.

23. The method of any one of claims 7-22, wherein n is 1.

24. The method of any one of claims 7-22, wherein n is 2.

25. The method of any one of claims 7-22, wherein n is 3.

26. The method of claim 7, wherein the dual inhibitor is selected from:

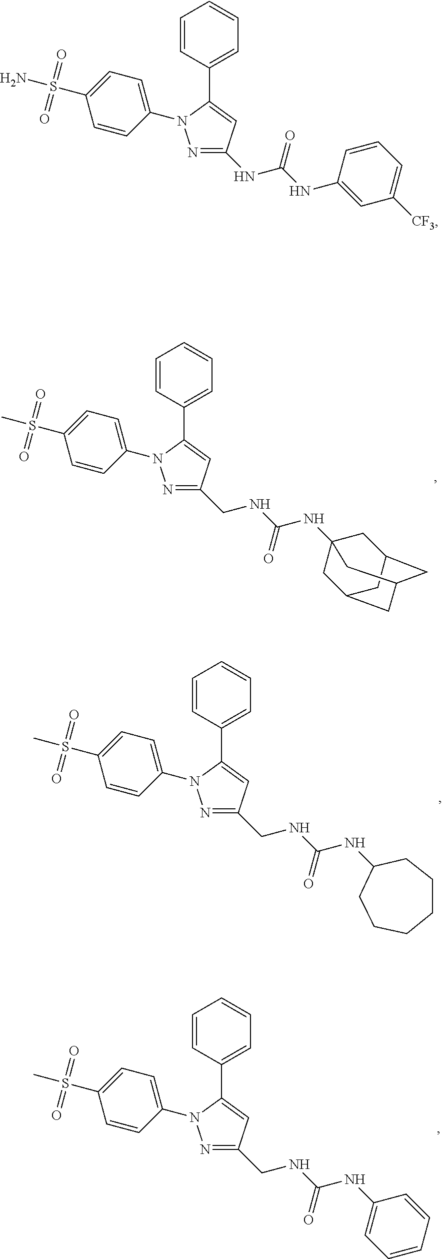 [SMILES: NS(=O)(=O)c1ccc(-n2nc(NC(=O)Nc3cccc(C(F)(F)F)c3)cc2-c2ccccc2)cc1 ; CS(=O)(=O)c1ccc(-n2nc(CNC(=O)NC34CC5CC(CC(C5)C3)C4)cc2-c2ccccc2)cc1 ; CS(=O)(=O)c1ccc(-n2nc(CNC(=O)NC3CCCCCC3)cc2-c2ccccc2)cc1 ; CS(=O)(=O)c1ccc(-n2nc(CNC(=O)Nc3ccccc3)cc2-c2ccccc2)cc1]

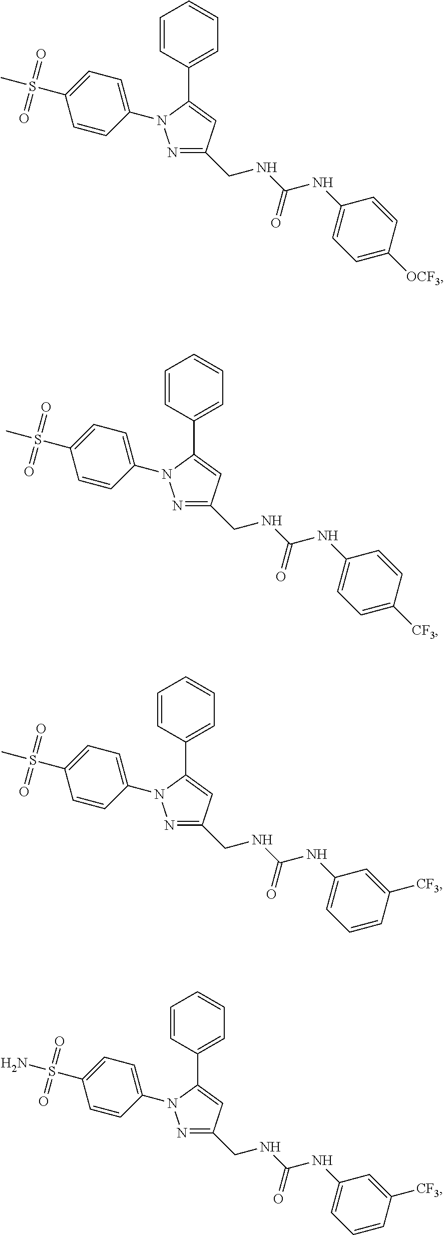 [SMILES: CS(=O)(=O)c1ccc(-n2nc(CNC(=O)Nc3ccc(OC(F)(F)F)cc3)cc2-c2ccccc2)cc1 ; CS(=O)(=O)c1ccc(-n2nc(CNC(=O)Nc3ccc(C(F)(F)F)cc3)cc2-c2ccccc2)cc1 ; CS(=O)(=O)c1ccc(-n2nc(CNC(=O)Nc3cccc(C(F)(F)F)c3)cc2-c2ccccc2)cc1 ; NS(=O)(=O)c1ccc(-n2nc(CNC(=O)Nc3cccc(C(F)(F)F)c3)cc2-c2ccccc2)cc1]

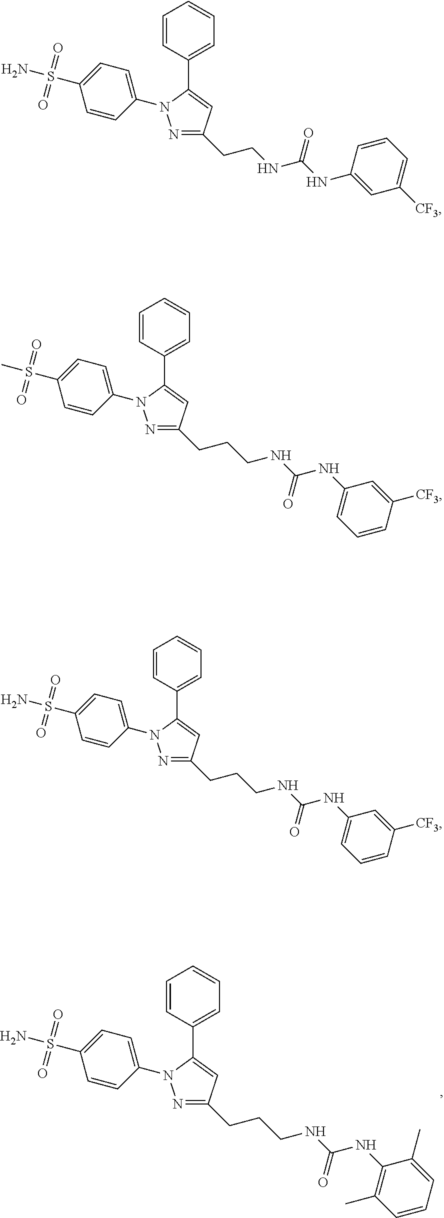 [SMILES: NS(=O)(=O)c1ccc(-n2nc(CCNC(=O)Nc3cccc(C(F)(F)F)c3)cc2-c2ccccc2)cc1 ; CS(=O)(=O)c1ccc(-n2nc(CCCNC(=O)Nc3cccc(C(F)(F)F)c3)cc2-c2ccccc2)cc1 ; NS(=O)(=O)c1ccc(-n2nc(CCCNC(=O)Nc3cccc(C(F)(F)F)c3)cc2-c2ccccc2)cc1 ; Cc1cccc(C)c1NC(=O)NCCCc1cc(-c2ccccc2)n(-c2ccc(S(N)(=O)=O)cc2)n1]

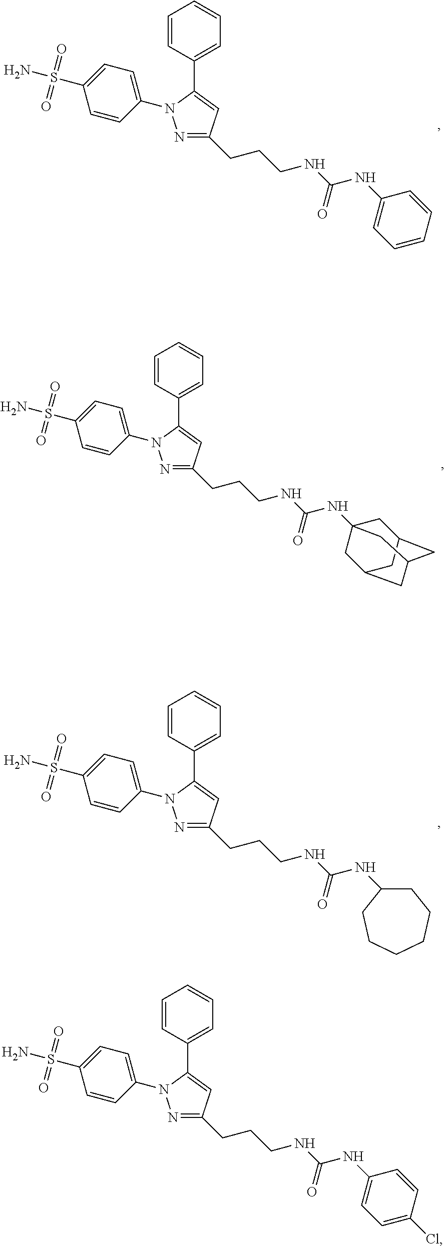 [SMILES: NS(=O)(=O)c1ccc(-n2nc(CCCNC(=O)Nc3ccccc3)cc2-c2ccccc2)cc1 ; NS(=O)(=O)c1ccc(-n2nc(CCCNC(=O)NC34CC5CC(CC(C5)C3)C4)cc2-c2ccccc2)cc1 ; NS(=O)(=O)c1ccc(-n2nc(CCCNC(=O)NC3CCCCCC3)cc2-c2ccccc2)cc1 ; NS(=O)(=O)c1ccc(-n2nc(CCCNC(=O)Nc3ccc(Cl)cc3)cc2-c2ccccc2)cc1]

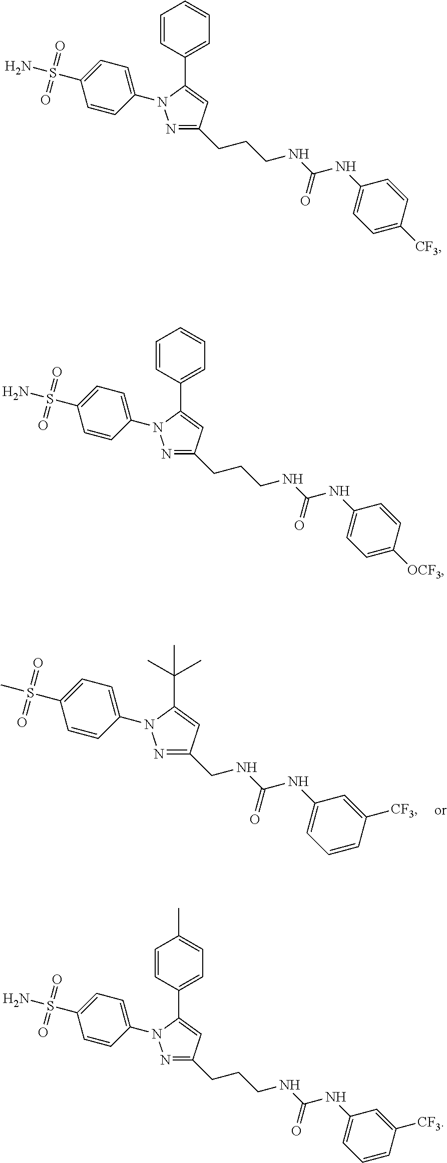 [SMILES: NS(=O)(=O)c1ccc(-n2nc(CCCNC(=O)Nc3ccc(C(F)(F)F)cc3)cc2-c2ccccc2)cc1 ; NS(=O)(=O)c1ccc(-n2nc(CCCNC(=O)Nc3ccc(OC(F)(F)F)cc3)cc2-c2ccccc2)cc1 ; CC(C)(C)c1cc(CNC(=O)Nc2cccc(C(F)(F)F)c2)nn1-c1ccc(S(C)(=O)=O)cc1 ; Cc1ccc(-c2cc(CCCNC(=O)Nc3cccc(C(F)(F)F)c3)nn2-c2ccc(S(N)(=O)=O)cc2)cc1]

27. The method of claim 7, wherein the dual inhibitor is

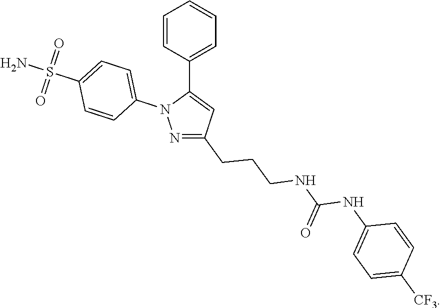 [SMILES: NS(=O)(=O)c1ccc(-n2nc(CCCNC(=O)Nc3ccc(C(F)(F)F)cc3)cc2-c2ccccc2)cc1]

28. The method of claim 7, wherein the dual inhibitor is PTUPB (OX1).

29. The method of any one of claims 1-28, wherein the disease associated with fibrotic progression is idiopathic pulmonary fibrosis.

30. PTUPB for use in the treatment of idiopathic pulmonary fibrosis.

31. A dual inhibitor capable of inhibiting sEH and a secondary target, wherein the dual inhibitor inhibits sEH with IC₅₀ of 1 micromolar or less, 100 nanomolar or less, 50 nanomolar or less, 10 nanomolar or less, 1 nanomolar or less, 50 picomolar or less, 10 picomolar or less, or 1 nanomolar or less.

32. The dual inhibitor of claim 31, wherein the dual inhibitor inhibits the secondary target with IC₅₀ of 1 micromolar or less, 100 nanomolar or less, 50 nanomolar or less, 10 nanomolar or less, 1 nanomolar or less, 50 picomolar or less, 10 picomolar or less, or 1 nanomolar or less.

33. The dual inhibitor of claim 31, wherein the dual inhibitor has a half-life in liver microsomes of at least five minutes, at least one hour, at least two hours, at least twelve hours, at least one day, at least two days, or at least one week.

34. A method for treating or preventing a disease associated with fibrotic progression in a subject in need thereof, comprising administering a compound whose structure comprises an inhibitor of sEH.

35. The method of claim 34, wherein the inhibitor of sEH is selected from the group consisting of PTUPB (OX1), Pirfenidone, Nintedanib, and GSK225629415 (OX3).

36. The method of claim 34, wherein the inhibitor of sEH is selected from the small molecules disclosed in US 2009/0023731.

37. The method of claim 34, wherein the inhibitor of sEH is selected from the small molecules disclosed in U.S. Pat. No. 8,815,951.
